(19) United States
(12) Patent Application Publication
(10) Pub. No.: US 2024/0368563 A1
(43) Pub. Date: Nov. 7, 2024

(54) IN VITRO METHOD OF INVASIVE AND DUCTAL CELL GROWTH

(71) Applicants: Helmholtz Zentrum München - Deutsches Forschungszentrum Für Gesundheit Und Umwelt (GmbH), Neuherberg (DE); Technische Universität München, München (DE)

(72) Inventors: Hilary GANZ, Wiesbaden (DE); Lisa ENGELBRECHT, München (DE); Christina SCHEEL, Recklinghausen (DE); Andreas BAUSCH, Unterschleissheim (DE); Benedikt BUCHMANN, München (DE)

(21) Appl. No.: 18/686,841

(22) PCT Filed: Aug. 26, 2022
(86) PCT No.: PCT/EP2022/073755

§ 371 Date: Feb. 26, 2024

(30) Foreign Application Priority Data
Aug. 27, 2021 (EP) 21193574.7

(51) Int. Cl. C12N 5/071; C12N 5/09

(52) U.S. Cl. CPC C12N 5/0697; C12N 5/0631; C12N 5/0693; C12N 2513/00; C12N 2533/54; C12N 2533/90

(57) ABSTRACT

The present invention relates to an in vitro method of generating cells capable of differentiating to a multicellular organoid unit that morphologically and/or functionally recapitulates invasive and/or ductal cell growth. The present invention further relates to a method of screening for an anti-migratory drug using a multicellular organoid unit obtained in the in vitro method. Additionally, the present invention relates to a culture medium and the respective use of said culture medium in any of said methods according to the present invention.

### TECHNICAL FIELD OF THE INVENTION

The present invention concerns an in vitro method of generating cells capable of differentiating to a multicellular organoid unit that morphologically and/or functionally recapitulates invasive and/or ductal cell growth. The present invention further relates to a method of screening for an anti-migratory drug using a multicellular organoid unit obtained in the in vitro method. Additionally, the present invention relates to a culture medium and the respective use of said culture medium in any of said methods according to the present invention.

### BACKGROUND OF THE INVENTION

The mammary gland consists of a bilayered branched epithelial network with an inner layer of luminal cells and an outer layer of myoepithelial/basal cells. Breast cancer is thought to arise generally from the luminal epithelial cells¹⁻⁴. Highly proliferative cancerous cells can either remain within the confined luminal spaces (in situ carcinomas) or breach the basal cell layer and lamina, resulting in invasive breast carcinomas⁵. There are several morphologically distinct types of invasive carcinomas. The most commonly diagnosed type is invasive carcinoma of no special type (NST), also known as invasive ductal carcinoma, which comprises over 70% of all cases⁶. Histological grade of NST is among other factors determined based on the extent to which cancer cells form differentiated ducts, a measurement that bears prognostic value⁷⁻¹⁰. Hereby, arising ducts resemble normal mammary gland morphology, however, they consist solely of invasive luminal cells that maintain expression of luminal markers such as GATA-3^{11,12} and specific cytokeratins¹³. In this grading system, a high degree of well differentiated ductal network formation is attributed to a low-grade of the disease¹⁰. Certain genetic aberrations appear to have direct impact on mammary duct formation. One prominent example is E-cadherin, which is typically lost in invasive lobular carcinomas (ILC), a subtype where duct formation is completely absent^{14,15}.

Understanding the mechanisms that underlie invasive outgrowth of epithelial cells represents the key to prevention of invasive breast cancer. Importantly, it has so far not been possible to identify universal genetic alterations that distinguish pre-invasive from invasive carcinomas¹⁶⁻¹⁸. Recent single cell analyses support these findings by showing that rather than being driven by one or a few dominant clones, a variety of clones invade concomitantly^{19,20}. This lack of support for specific genetic determinants of invasion might indicate that a conserved mechanism for invasive growth is already present within pre-invasive luminal cells and that the process of invasion is driven by the microenvironment or tumor stroma^{21,22}. This fits with the perception that basal cells and basal lamina act as a physical, invasion-suppressing barrier between luminal cells and ECM²³⁻²⁵. However, during invasive cancer development, this barrier loses its integrity allowing direct contact of luminal cells to the ECM^{23,24,26,27}. The main structural component of the human mammary ECM is collagen type I, which is even more predominant in invasive carcinomas^{28,29}. In vivo, collagen type I can drive tumor cell invasion³⁰⁻³². In addition, collagen type I has been shown to encourage invasive behavior and support branching morphogenesis in vitro in normal epithelial cells including the basal mammary epithelial subset³³⁻³⁵.

In analogy, invasive growth could be elicited from luminal cells once they converted towards a basal phenotype⁶⁷. However, collagen-induced invasive branching morphogenesis resulting in the ductal morphology that is characteristic for NST carcinomas has so far not been observed in luminal cells in vitro. Instead, human luminal progenitor (LP) cells cultivated in collagen type I gels in a manner where their luminal identity was maintained were shown to grow out into spheres or budding-like structures³⁵ with reversed apical-basal polarity³⁶. Consequently, 3D models of invasive growth have relied mainly on in vitro immortalized mammary epithelial or breast cancer-derived cell lines^{37,38} that often lack luminal markers and do not reflect the morphogenetic aspects of invasion³⁹⁻⁴¹. Presumably, one reason for the lack of luminal cell-based models is that maintaining viable and actively dividing luminal cells in culture has been an ever-present challenge.

The luminal cells of the breast are the cells of origin for almost all types of breast cancer. During breast cancer progression, highly proliferative luminal cells can start growing out of their initial ductal network and invade the surrounding extracellular matrix. Thus, the cancer becomes invasive. Invasion is the foundation for breast cancer metastases and the cause for over 90% of cancer related death. Despite the impact of cancer cell invasion on the prognosis of all solid tumors, most chemotherapies rely on cytostatic drugs aimed towards a reduction of proliferation within tumor cells. However, this unspecific and cytotoxic treatment results in the well-known side effects of chemotherapy such as fatigue, nausea, bone marrow suppression and alopecia. A more specific therapy targeting invasive and migratory behavior is therefore of great interest. Consequently, the interest in drugs targeting invasive and migratory aspects of cancer progression is on the rise. Suitable pre-clinical models are an essential tool for efficient drug development. However, the possibilities for modelling this invasive process through the extracellular matrix (ECM) performed by luminal cells are so far very limited. Invasion from primary human luminal cells could never be recreated in vitro, because luminal cells simply never showed the strong invasive capacity that would be expected for this subset based on its in vivo capacity. Therefore, the knowledge on luminal cell invasion in the human mammary gland is limited to histopathological sections that have been removed from a diseased mammary gland. What these sections clearly show is that particularly early luminal cell invasion is a highly organized process as cells form a branched network of ducts, morphologically similar to the normal mammary gland. While the information obtained through these sections are valuable, a dynamic and accessible model for the actual invasive branching morphogenesis process resulting in this phenotype is lacking so far.

Another noteworthy emerging problem in primary cell research of breast cancer processes is neoadjuvant chemotherapy, which is by now the standard approach in breast cancer treatment and increasingly limits access to untreated tumor tissue that could be used to establish in vitro models.

Consequently, there is an unsatisfied need for making available and providing luminal cells, especially from a healthy source, and respective conditions, assays and in vitro methods and models that are able to resemble and recapitulate invasive and/or ductal cell growth, luminal cell invasion and disease-development. Therefore, the purpose of the invention is to accurately model in vivo invasive processes in an in vitro assay that can be observed and manipulated easily. Therefore, the inventors of the present invention designed a set-up, in which luminal progenitor (LP) cells, which are the actual cell type that undergoes these invasive processes in vivo, grow invasively in vitro.

### SUMMARY OF THE INVENTION

The present invention relates to an in vitro method of generating cells capable of differentiating to a multicellular organoid unit that morphologically and/or functionally recapitulates invasive and/or ductal cell growth, comprising the steps of: (i) Providing luminal progenitor (LP) cells; (ii) culturing said luminal progenitor (LP) cells in a collagen gel in a culture medium for at least 5 days, wherein said culture medium comprises one or more growth factor(s), one or more inhibitor(s) of cell contractility and one or more serum component or serum substitute, and (iii) determining whether a multicellular organoid unit is formed in step (ii).

In one embodiment, it is preferred for said in vitro method that the multicellular organoid unit is a multicellular breast organoid unit.

In one embodiment, it is preferred for said in vitro method that determining whether a multicellular organoid unit is formed is by determining whether an invasive organoid or a ductal structure or one or more branch-point(s) is/are comprised in said multicellular organoid unit.

In one preferred embodiment of the in vitro method of the present invention, the provided luminal progenitor (LP) cells are human primary luminal cells or human mammary luminal progenitor cells, preferably healthy human primary luminal cells or healthy human mammary luminal progenitor cells.

It is further preferred for the in vitro method of the present invention that the one or more growth factor(s) of the culture medium is a ligand of the ErbB receptor family, preferably the epidermal growth factor (EGF) or an analogue thereof.

The in vitro method of the present invention further comprises that in one preferred embodiment the one or more inhibitor(s) of cell contractility of the culture medium is a Rho-kinase (ROCK) inhibitor, preferably Y-27632 or thiazovivin, or a myosin inhibitor, preferably para-amino blebbistatin.

In one preferred embodiment of the in vitro method of the present invention, the culture medium further comprises one or more selected from the group consisting of N-acetylcysteine, neuregulin 1, a vitamin, preferably nicotinamide, an antibiotic, a fibroblast growth factor (FGF), preferably FGF7 or FGF10, a MAP kinase inhibitor, preferably SB202190, a supplement and a buffer.

In one further preferred embodiment of the in vitro method of the present invention, the provided luminal progenitor (LP) cells are genetically modified, preferably one or more gene(s) are knocked-out in the provided luminal progenitor (LP) cells.

The in vitro method of the present invention further comprises that in one preferred embodiment the collagen concentration of the collagen gel is in a range of about 0.5 mg/ml to about 3 mg/ml, preferably of about 0.8 mg/ml to about 2.0 mg/ml, more preferably of about 1.0 mg/ml to about 1.5 mg/ml.

In one preferred embodiment of the in vitro method of the present invention, the multicellular organoid unit morphologically and/or functionally recapitulates low-grade invasive processes of mammary cancer, preferably of low-grade carcinoma of no special type (NST).

The present invention further relates to a method of screening for an anti-migratory drug, comprising the following steps: a) Bringing a multicellular organoid unit obtained by the in vitro method according to the present invention into contact with a compound suspected of being an anti-migratory drug, b) determining whether or not said compound elicits a cellular response in the multicellular organoid unit, with the proviso that when said compound elicits a cellular response compared to a reference state, said compound is an anti-migratory drug.

In one preferred embodiment of the method of screening of the present invention, the cellular response is selected from the group consisting of cell elongation arrest, cell proliferation arrest, growth arrest, apoptosis, necrosis, DNA damage, inhibition of differentiation, migration arrest, and changes in the morphology of cells, preferably cell elongation arrest.

The present invention also relates to a culture medium comprising:
one or more growth factor(s),
one or more inhibitor(s) of cell contractility,
one or more serum component or serum substitute, and
further one ore more of the group consisting of N-acetylcysteine, neuregulin 1, a vitamin, preferably nicotinamide, an antibiotic, a fibroblast growth factor (FGF), preferably FGF7 or FGF10, a MAP kinase inhibitor, preferably SB202190, a supplement and a buffer.

In one preferred embodiment of the culture medium according to the present invention, the one or more growth factor(s) is a ligand of the ErbB receptor family, preferably the epidermal growth factor (EGF) or an analogue thereof.

In one preferred embodiment of the culture medium according to the present invention, the one or more inhibitor(s) of cell contractility is a Rho-kinase (ROCK) inhibitor, preferably Y-27632 or thiazovivin, or a myosin inhibitor, preferably para-amino blebbistatin.

Additionally, the present invention also relates to the use of a culture medium according to the present invention as described herein in any of said methods according to the present invention as described herein.

### BRIEF DESCRIPTION OF THE FIGURES

FIG. 1 shows that human mammary luminal progenitor cells give rise to complex branched ductal structures in collagen type I gels. FIG. 1A shows normal mammary gland morphology, namely a schematic illustration and a p63 (brown) stained section of normal mammary ducts. Scale bar: 50 μm. FIG. 1B shows sorting of epithelial cells from reduction mammoplasties via FACS based on EpCAM and CD49f expression. The green circle indicates the CD49f^{high}/EpCAM^{high} population (=luminal progenitors (LP)). FIG. 1C shows the experimental set-up with carmine staining of arising structure types in BLOM-medium. Scale bar: 100 μm. FIG. 1D shows quantification of outgrowing structure types in BLOM-medium. Depicted are donors M35, M44 and M45. The structure type was normalized to total structures arising per gel. Data are mean±s.d. n=4 gels/condition. FIG. 1E shows carmine stainings of branched structures arising from donors M16, M28, M35, M36, M42 and M44 in BLOM-medium. Scale bar: 100 μm. FIG. 1F shows the schematic illustration of invasively growing LP cells and an H&E stained section of low-grade NST carcinoma. Scale bar: 50 μm.

FIG. 2 shows that human mammary luminal progenitor cells give rise to complex branched ductal structures in collagen type I gels. FIG. 2A shows an extended FACS sorting layout of normal human mammary epithelial cells (S=stroma, LM=luminal mature, LP=luminal progenitor, B=basal). FIG. 2B shows reanalysis of FACS sorted populations. Cross contamination was calculated in % of all CD31⁻/CD45⁻ cells. FIG. 2C shows carmine staining of arising structure types in BCOM-medium. Scale bar: 100 μm. FIG. 2D shows quantification of outgrowing structure type in BCOM-medium. Depicted are donors M35, M44 and M45. The structure type was normalized to total structures arising per gel. Data are mean±s.d. n=4 gels/condition. FIG. 2E shows a light microscopy picture of LP cells in BLOM-medium in Matrigel matrix after 7d in culture. Scale bar: 100 μm. FIG. 2F shows carmine staining of structure formation by luminal mature (CD49f^{low}/EpCAM^{high}) and basal (CD49f^{high}/EpCAM^{low}/CD10⁺) cells. Scale bar: 100 μm.

FIG. 3 shows that branched luminal organoids arise clonally and express luminal lineage and polarization markers. FIG. 3A shows light microscopy pictures of a branched luminal organoid developing from a single cell over the course of 12 d. Scale bar: 100 μm. FIG. 3B shows a schematic illustration of invasively growing LP cells and p63 (brown) or GATA3 (brown) stained section of ducts arising in low-grade NST carcinomas. Scale bar: 50 μm. FIGS. 3C-H show confocal microscopy on branched LP-derived organoids: representative images of luminal and polarization markers. CK8/18 (green), GATA3 (green), ZO-1 (green), Muc1 (green), Laminin (green), F-Actin (white), DAPI (blue). n=30 structures/condition; 3 donors; 10 structures/donor. Scale bar: 50 μm. FIG. 3I shows quantification of ducts with and without correctly polarized expression of markers. Data are presented as stacked bars with mean±s.d. n=3 donors, 25 ducts per donor.

FIG. 4 shows that branched luminal organoids are negative for basal lineage marker. FIGS. 4A/C show confocal microscopy on branched basal-cell derived branched structures: Representative images of basal lineage marker expression. α-SMA (red), p63 (red), DAPI (blue). Scale bar: 50 μm. n=30 structures/condition; 3 donors; 10 structures/donor. FIGS. 4B/D show confocal microscopy on branched LP-derived branched structures: Representative images of basal lineage marker expression. α-SMA (red), p63 (red), DAPI (blue). Scale bar: 50 μm. n=30 structures/condition; 3 donors; 10 structures/donor.

FIG. 5 shows that LP-derived branched organoid formation requires inhibition of ROCK-myosin II signaling. FIG. 5A shows carmine stainings of branched structures arising in BLOM-medium with Y-27632 or without (DMSO ctrl). Scale bar: 100 μm. FIG. 5B shows quantification of branched structures in BLOM-medium with Y-27632 or without (DMSO ctrl). Depicted are donors M35, M44 and M45. Branched structures were normalized to total structures arising per gel. Data are mean±s.d. n=4 gels/condition. P values were calculated using an unpaired two-tailed t-test, **P≤0.01, ***P≤0.001, ****P≤0.0001. FIG. 5C shows quantification of branched structure sizes in BLOM-medium with Y-27632 or without (DMSO ctrl). Depicted are donors M35, M44 and M45. Size was defined as the area covered by the organoid when all tips were connected virtually using ImageJ. Data are shown as median±25%. n=4 gels/condition. P values were calculated using an unpaired two-tailed t-test, *P≤0.05, **P≤0.01, ****P≤0.0001. FIG. 5D shows confocal microscopy on branched luminal organoids grown in BLOM-medium with Y-27632 or without (DMSO ctrl): representative images of polarization markers. Laminin (green), F-Actin (white), DAPI (blue). Scale bar: 50 μm. FIG. 5E shows at the top: Directionality of cellular movement calculated as quotient of velocity directed in parallel to extending duct (V_{II}) and orthogonal to it (V_{I}). Data are shown as median ±25%. P value was calculated using an unpaired two-tailed t-test, ****P≤0.0001. At the bottom is shown a schematic depiction of elongating duct. FIG. 5F/G show displacement of fluorescent beads relative to t=0 measured by live cell imaging with Y-27632 or without (DMSO ctrl). T=0 hrs (blue) vs. t=0<t≤18/19 hrs (green). Scale bar: 200 μm. Experiments were repeated in n=3 donors with a total of individual organoids n≥30.

FIG. 6 shows that LP-derived branched organoid formation requires inhibition of ROCK-myosin II signaling. FIG. 6A shows quantification of spheres and sticks in BLOM-medium with Y-27632 or without (DMSO ctrl). Depicted are donors M35, M44 and M45. Counts were normalized to total structures arising per gel. Data are mean±s.d. n=4 gels/condition. P values were calculated using an unpaired two-tailed t-test, n.s=P≥0.05, **P≤0.01, ***P≤0.001, ****P≤0.0001. FIG. 6B shows quantification of branched structure complexity in BLOM-medium with Y-27632 or without (DMSO ctrl). Depicted are donors M35, M44 and M45. Branched structure types were normalized to total branched structures arising per gel. Data are mean±s.d. n=3 gels/condition. P values were calculated using an unpaired two-tailed t-test, n.s.=P≥0.05, **P≤0.01, ***P≤0.001, ****P≤0.0001. FIG. 6C shows carmine stainings of branched structures arising in BLOM-medium with Y-27632 or without Y-27632. In conditions without Y-27632, either DMSO or PAB was added. Scale bar: 100 μm. FIG. 6D shows quantification of branched structures in Y-27632 or without Y-27632. In conditions without Y-27632, either DMSO or PAB was added. Branched structure formation was normalized to BLOM-medium with Y-27632. Data are mean±s.d. n=3 independent experiments with donors M16, M28 and M44. P values were calculated using an unpaired two-tailed t-test, n.s.=P≥0.05, *P≤0.05, **P≤0.01.

FIG. 7 shows that ductal organoids are generated through invasive branching morphogenesis. FIG. 7A shows alignment of collagen fibers in control sections of the gel without cells vs. in front of invading ducts. Scale bar: 50 μm. n=22 individual organoids. FIG. 7B shows duct elongation upon treatment with Marimastat vs. control organoids measured by live cell imaging with t=0 hrs (black) vs. t=17 hrs (red). Scale bar: 200 μm. n=18 individual organoids. FIG. 7C shows confocal microscopy on tip cell F-Actin (white), DAPI (blue). Scale bar: 50 μm. FIG. 7D shows tip cell exchange. Cells tracked by live cell imaging. Tip cell 1 (green arrow), tip cell 2 (blue arrow). Organoids measured by live cell imaging via nuclear labelling with sirDNA (white). Scale bar: 50 μm. FIG. 7E shows bifurcation at tip. Organoids measured by live cell imaging via nuclear labelling with sirDNA (pink). Scale bar: 80 μm. FIG. 7F shows confocal microscopy on invading organoid. E-Cadherin (green), DAPI (blue). Scale bar: 50 μm. FIG. 7G shows duct elongation of organoid (day 8) upon addition of HECD1 antibody on day 5. Organoids measured by live cell imaging over the course of 20 hrs via nuclear labelling with sirDNA (white). Scale bar: 50 μm. n=30 individual organoids. All experiments were repeated in n=3 donors.

FIG. 8 shows that ductal organoids are generated through invasive branching morphogenesis. FIG. 8A shows quantification of fiber alignment degree in control sections of the gel without cells vs. in front of invading ducts. Data are shown as median±25%. P value was calculated using an unpaired two-tailed t-test, ****P≤0.0001. n=22 individual organoids. FIG. 8B shows quantification of duct elongation upon treatment with Marimastat vs. control organoids tracked by live cell imaging. Data are shown as median±25%. P value was calculated using an unpaired two-tailed t-test, ****P≤0.0001. n=18 individual organoids. FIG. 8C shows confocal microscopy on branched LP-derived organoids: representative images of Laminin (green), and DAPI (blue) on Day 9 and Day 13. N=30 structures/condition; 3 donors; 10 structures/donor. Scale bar: 50 μm. FIG. 8D shows quantification of ducts with and without expression of correctly polarized laminin on Day 9 as compared to Day 13. Data are presented as stacked bars with mean #s.d. n=3 donors, 25 ducts per donor. FIG. 8E shows movement of tip cells over the course of 0-400 min measured by live cell imaging via nuclear labelling with sirDNA (white). Scale bar: 200 μm. FIG. 8F shows an image of the organoid on day 9 after HECD1 treatment from day 5 on. Nuclear labelling with sirDNA (pink). Scale bar: 100 μm. All experiments were repeated in n=3 donors.

FIG. 9 shows that deletion of E-cadherin results in ILC-like morphology. FIG. 9A shows immunofluorescence of E-cadherin expression in 2D culture and schematic illustration of clonal outgrowth in 3D culture upon transfection and enrichment. E-cadherin (green), DAPI (blue). Scale bar: 50 μm. FIG. 9B shows modification efficiency on the protein level. All structures, found in gels after transfection and enrichment, were classified via confocal microscopy by E-cadherin status into wt vs. KO structures. Depicted are donors M16, M44 and M45. Data are presented as stacked bars and values were normalized to the total number of structures per condition. FIG. 9C shows quantification of the structure type arising in collagen gels after transfection and enrichment divided by E-cadherin status of the structures (wt vs. KO). Depicted are donors M16, M44 and M45. Data are presented as stacked bars and each structure category is normalized to the total number of structures per condition. FIG. 9D shows quantification of maximum width of arising branched structures and sticks. Structures are divided by E-cadherin status into wt vs. KO. Data are shown as median±25%. P values were calculated using an unpaired two-tailed t-test, ****P≤0.0001. FIG. 9E shows confocal microscopy of structures growing out in collagen gels after transfection and enrichment that either still maintained E-cadherin (wt) or had lost it (KO). E-cadherin (green), CK8/18 (red), DAPI (blue). Scale bar: 50 μm. FIG. 9F shows H&E and E-cadherin (brown) stained sections of NST carcinoma and ILC. Scale bar: 50 μm.

FIG. 10 shows that deletion of E-cadherin results in ILC-like morphology. FIG. 10A shows branched structure formation capacity upon passaging in 2D culture. Scale bar: 100 μm. FIG. 10B shows the sequence of the E-cadherin encoding gene CDH1. Exons are depicted in pink. gRNAs are depicted in blue. Schematic target sites of the two gRNAs on the gene are shown. FIG. 10C shows modification efficiency in the bulk population of transfected LP cells on the genetic level vs. wt cells. FIG. 10D shows confocal microscopy of structures growing out in collagen gels after transfection and enrichment that either still maintained E-cadherin (wt) or had lost it (KO). E-cadherin (green), Ki-67 (red), DAPI (blue). Scale bar: 50 μm. FIG. 10E shows quantification of percentage of Ki-67 positive cells within a structure in wt as compared to KO structures. Data are shown as median±25%. n=10 structures. P values were calculated using an unpaired two-tailed t-test, n.s.=P≥0.05.

### DETAILED DESCRIPTION OF THE INVENTION

The inventors of the present invention were able to recapitulate invasive capacity of primary human LP cells in a collagen gel, preferably in a collagen type I gel/matrix. In detail, the inventors of the present invention have developed specific 3D culture conditions, in which single LP cells gave rise to a multicellular branched ductal network resembling/recapitulating the morphology of e.g. low-grade NST without requiring any genetic perturbation to do so. Thereby, the inventors found that the invasive branching morphogenesis process may be enabled by matrix remodeling leader cells and can additionally comprise inhibition of the Rho-ROCK-Myosin II signaling cascade.

The inventors observed that in one embodiment, CRISPR-Cas9 mediated deletion of E-cadherin in healthy luminal cells resulted in diffusely invading organoids resembling ILC morphology. In detail, in said embodiment, the inventors showed that knockout of E-cadherin with the CRISPR-Cas9 system causes dissolution of duct formation as observed in e.g. invasive lobular carcinoma (ILC), a subtype of invasive carcinomas where E-cadherin function is frequently lost.

Together, the present invention shows that invasive branching morphogenesis, resulting in one embodiment in low-grade NST morphology, is an innate cellular program that can be triggered by specific growth conditions within healthy LP cells in vitro. Thus, the inventors of the present invention developed an in vitro method, respectively model or assay, for primary human LP cells, in which single cells of preferably healthy origin give rise to complex branched structures resembling and recapitulating the ductal morphology of e.g. low-grade carcinoma of no special type (NST). Thereby, the inventors of the present invention inter alia identified e.g. reduced actomyosin contractility for collective invasion via matrix-remodeling leader cells. The in vitro method of the present invention shows that invasive capacity can be elicited from healthy luminal cells in specific environments, which results for example in low-grade NST morphology. Thereby, this method of the present invention offers a platform to investigate the dynamics of luminal cell invasion and unravel the impact of genetic aberrations on invasive morphology.

Thus, the present invention relates to an in vitro method of generating cells capable of differentiating to a multicellular organoid unit that morphologically and/or functionally recapitulates invasive and/or ductal cell growth, comprising the steps of:
(i) Providing luminal progenitor (LP) cells;
(ii) culturing said luminal progenitor (LP) cells in a collagen gel in a culture medium for at least 5 days,
   wherein said culture medium comprises
   one or more growth factor(s),
   one or more inhibitor(s) of cell contractility and
   one or more serum component or serum substitute, and
(iii) determining whether a multicellular organoid unit is formed in step (ii).

In the context of the present invention, the term “morphologically” relates to the study of the form and/or structure of e.g. an organism or part thereof, especially to study the form and/or structure of the generated cells, the differentiated cells or the formed multicellular organoid unit according to the method of the present invention. In this regard, the term “morphologically recapitulates” means that the investigated cells or the multicellular organoid unit is respectively studied concerning structure and form and it is examined if the detected structure or form of the generated cells or of the generated multicellular organoid unit has the same or similar morphology/form/structure as the state or condition the present invention aims at recapitulating, namely of invasive and/or ductal cell growth.

The term “functionally” relates to being of, being connected with, comprising or being a certain function, especially in the context of the present invention, a function that relates to invasive and/or ductal cell growth. In this regard, the term “functionally recapitulates” means that the investigated cells or the multicellular organoid unit is studied concerning function and that a function of the generated cells or the generated multicellular organoid unit is detected that has the same or a similar function as the state or condition the present invention aims at recapitulating, namely invasive and/or ductal cell growth.

In the context of the present invention, the term “invasive cell growth” means that the cells show invasion or characteristics of invasion, which is the direct extension and penetration by cancer cells into neighboring tissue. Invasion is generally distinguished from metastasis, which is the spread of cancer cells through the circulatory system or the lymphatic system to more distant locations. There exists a classification of invasive growth types concerning breast cancer, which is as follows: Despite the considerable structural diversity of the primary breast tumor, five main types of morphological structures can be distinguished: alveolar, trabecular, tubular and solid structures, and discrete groups of tumor cells. The alveolar structures are tumor cell clusters of round or slightly irregular shape. The morphology of the cells that form this type of structures varies from small cells with moderate cytoplasm and round nuclei to large cells with hyperchromatic nuclei of irregular shape and moderate cytoplasm. The trabecular structures are either short, linear associations formed by a single row of small, rather monomorphic cells or wide cell clusters consisting of two rows of medium-sized cells with moderate cytoplasm and round normochromic or hyperchromatic nuclei. The tubular structures are formed by a single or two rows of rather monomorphic cells with round normochromic nuclei. The solid structures are fields of various sizes and shapes, consisting of either small cells with moderate cytoplasm and monomorphic nuclei or large cells with abundant cytoplasm and polymorphic nuclei. Discrete groups of cells occur in the form of clusters of one to four cells with variable morphologies.

In the context of the present invention, the term “ductal cell growth” means that cells, in particular cancer cells grow into partly hollow and polarized tube-like forms. Thereby, in case of cancer-like growth, the ducts grow outside of the normal mammary gland ducts into other parts of the breast tissue. In contrast to the typically bi-layered epithelium of the normal mammary gland consisting of basal and luminal cells, in cancer-like ductal growth, generated ducts only consist of one cell layer.

The term “one or more growth factors” according to the present invention may mean proteins that bind to receptors on the cell surface, with the primary result of activating a signal transduction cascade, and eventually influencing cellular proliferation, differentiation, or apoptosis. Growth factors include, for example, the following families: EGF (Epidermal Growth Factors), IGF (Insulin-like growth Factors), FGF (Fibroblast Growth Factors), Wnt (Wingless), TGF-beta (Transforming Growth Factor beta), Notch, and shh (sonic hedgehog).

In the context of the present invention, the term “inhibitor of cell contractility” means a substance, compound or protein that is able to inhibit, to modify or to reduce cell contractility of one or more cells or of one or more differentiated cells or of the multicellular organoid unit of the present invention. Thus, this also comprises that the cell contractility is modified by such an inhibitor that cell contractility is chanced compared to the state without said inhibitor, which is the reference state in this case. Especially, in one preferred embodiment, cell contractility is so modified that it is reduced compared to the respective reference state, which is without the mentioned inhibitor.

As used herein, the term “serum component” means a component, compound or substance, which may be the fluid and solute component of blood, which does not play a role in coagulation. Respectively, the term “serum substitute” means any component, compound or substance, which can recapitulate the function or effects gained by any serum component.

Thus, the present inventors pioneered in providing an assay, which can be used interchangeably with the terms in vitro method or in vitro model herein, and that enables luminal progenitor (LP) cells and respectively the multicellular organoid unit build by these cells, to recapitulate invasive and/or ductal cell growth, luminal cell invasion and disease-development. In particular, the present inventors have developed means and methods, i.a. culturing conditions, that allow cells to form structures that resemble invasive and/or ductal cell growth as defined above, especially mammary luminal cell invasion, more specifically breast cancer.

The means and methods provided herein enable detection, isolation and manipulation of luminal progenitor cells, populations thereof as well as multicellular organoid units comprising these cells and studying of key aspects of tissue architecture and function thereof. The in vitro method and assay is highly quantitative and scalable, and provides a highly sensitive and specific, thus reproducible functional readout that is suitable for high-throughput screening.

In step (i) of the above-described in vitro method of the present invention, luminal progenitor cells are provided. It is in general conceivable to use cells obtained from any of a wide variety of sources, e.g. the cells may be single human luminal progenitor (LP) cells as cellular starting material. As used herein, the term “providing luminal progenitor (LP) cells” may mean that single LP cells are isolated from frozen human breast fragments using FACS. For example, it may be comprised by step (i) in one embodiment to collect human breast tissue from women that have undergone reductive breast surgery for cosmetic reasons. However, human breast tissue is also commercially available.

In one preferred embodiment, single human luminal progenitor (LP) cells may be used as cellular starting material. For a long time period, difficulties in propagation of those cells in vitro have hampered their utilization in model systems. The LP cells used in the in vitro method of the present invention may be preferably of healthy origin and may therefore be a highly available primary material. However, in one embodiment of the present invention, the LP cells used may also be from a diseased donor or origin, e.g. cancerous origin.

LPs are the cells of origin for almost all breast cancers and therefore the cell type that undergoes migratory and invasive processes in breast cancer development. Interestingly, it has been shown that in vivo an invasively growing LP cell is despite their divergent behavior per se not genetically different from a normal LP cell. Therefore, invasive capacity is supposedly a feature already present within pre-invasive luminal cells of healthy origin. Nevertheless, triggering invasive behavior in LP cells has previously never been possible in vitro for neither LP cells of healthy nor cancerous origin, which adds to the reasons why those cells have not been employed for breast cancer modelling so far.

As luminal progenitor cells may be the cells-of-origin for breast cancer, in contrast to breast stem cells, luminal progenitor cells typically form spheres, when cultured in a collagen gel. Luminal progenitor cells can however also result in the generation of branched structures, in particular multicellular organoid units as defined herein, in a collagen gel, when applying the in vitro method of the present invention. In this regard, luminal markers may remain unchanged.

Furthermore, in one embodiment, the luminal progenitor cells may be dissociated cells from mammary epithelial tissue, wherein said epithelial tissue is healthy or diseased tissue and/or wherein said diseased mammary epithelial tissue comprises germline or somatic mutations.

Luminal progenitor cells, obtainable as described herein, can be used for testing a compound, such as a drug, hormone, growth factor, antibody, nucleotide molecule, peptide, protein or (co-cultured) cell and others. Upon treatment, the luminal progenitor cells may show a cellular response, e.g., cell elongation arrest, cell proliferation arrest, growth arrest, apoptosis, necrosis, DNA damage, inhibition of differentiation, migration arrest, and changes in the morphology of cells. The luminal progenitor cells can thus be used as a tool for testing compounds for their potential to modulate cellular responses as described herein. Provided herein is therefore also the use of luminal progenitor cells for testing compounds, e.g. for their potential to induce or inhibit differentiation and/or de-differentiation, thereby e.g. assessing their carcinogenic potential. For example, compounds capable of inhibiting differentiation and/or inducing de-differentiation may be potentially cancerogenous compounds. Methods for determining the cellular responses such as differentiation and de-differentiation are well-known in the art and include, e.g., microscopy, PCR techniques such as real-time PCR or digital PCR, cell sorting/flow cytometry, immunocytochemistry, western blotting, and biomarker analysis.

In one preferred embodiment of the in vitro method of the present invention, the provided luminal progenitor (LP) cells are human primary luminal cells or human mammary luminal progenitor cells, preferably healthy human primary luminal cells or healthy human mammary luminal progenitor cells.

For example, primary human mammary luminal progenitor cells can be derived from fresh breast reduction tissue (reduction mammoplasty) by mechanical and/or enzymatic dissociation and, if desired, can be further purified by methods such as fluorescence activated cell sorting (FACS).

It is envisaged that “cells capable of differentiating” and thus “dissociated cells” are derived from healthy or diseased mammary luminal progenitor cells or tissue. “Diseased tissue” in particular refers to tissue comprising cells with germline or somatic mutations, e.g. in proto-oncogenes. The term includes tissue comprising cancerous and/or pre-cancerous cells and/or tissue derived from a patient diagnosed with breast cancer. “Healthy tissue”, on the other hand, refers to tissue from healthy donors that preferably do not comprise germline or somatic mutations, cancerous and/or pre-cancerous cells.

In order to obtain dissociated cells according to the present invention, luminal progenitor cells can be dissociated mechanically and/or enzymatically. Means and methods for mechanical and enzymatical tissue dissociation are well-known in the art. E.g., the tissue can be minced using scalpels or other suitable tools. Other means of mechanical tissue dissociation are also conceivable, e.g. sonication or others. Further, tissue dissociating agents may be used, typically including tissue degrading enzymes such as collagenase, trypsin, neutral protease or dispase, and other proteolytic enzymes. However, the tissue dissociating agents are not necessarily limited to enzymes. Other examples of tissue dissociating agents are chelating agents. The length of time required for treatment will vary depending on the sonication frequency, type of the agent, the concentration of agent, and the temperature at which treatment is conducted. Treatment is allowed to proceed until a sufficient amount of tissue has dissociated without causing undue damage to released cells or cellular aggregates.

The method of the invention may further comprise a step of culturing the dissociated cells in 2D-culture (or other methods) prior to transferring them to collagen gels. This step is also referred to as “pre-cultivation” herein and may be carried out between steps (i) and (ii) of the in vitro method of the present invention.

Without wishing to be bound by theory, it is thought that 2D-pre-cultivation may increase the ability of luminal progenitor cells to form multicellular organoid units. Pre-cultivation, in particular 2D pre-cultivation, further allows genetic manipulation of the cells prior to cultivation in the collagen gel. Pre-cultivation can be accomplished using standard protocols known in the art, depending on the type of cell, length of cultivation, desired cell morphology as well as density and other parameters.

Next, dissociated cells are plated in collagen gels. In one embodiment of step (ii) of the in vitro method of the present invention, culturing said luminal progenitor (LP) cells in a collagen gel is carried out in a culture medium for at least 5 days, at least 6 days, at least 7 days, at least 8 days, at least 9 days, at least 10 days, at least 11 days, at least 12 days, at least 13 days or at least 14 days. In one embodiment of step (ii) of the in vitro method of the present invention, culturing said luminal progenitor (LP) cells in a collagen gel is carried out in a culture medium for at least 6 days. In one embodiment of step (ii) of the in vitro method of the present invention, culturing said luminal progenitor (LP) cells in a collagen gel is carried out in a culture medium for at least 7 days. In one embodiment of step (ii) of the in vitro method of the present invention, culturing said luminal progenitor (LP) cells in a collagen gel is carried out in a culture medium for at least 8 days. In one embodiment of step (ii) of the in vitro method of the present invention, culturing said luminal progenitor (LP) cells in a collagen gel is carried out in a culture medium for at least 9 days. In one embodiment of step (ii) of the in vitro method of the present invention, culturing said luminal progenitor (LP) cells in a collagen gel is carried out in a culture medium for at least 10 days. In one embodiment of step (ii) of the in vitro method of the present invention, culturing said luminal progenitor (LP) cells in a collagen gel is carried out in a culture medium for at least 11 days. In one embodiment of step (ii) of the in vitro method of the present invention, culturing said luminal progenitor (LP) cells in a collagen gel is carried out in a culture medium for at least 12 days. In one embodiment of step (ii) of the in vitro method of the present invention, culturing said luminal progenitor (LP) cells in a collagen gel is carried out in a culture medium for at least 13 days. In one embodiment of step (ii) of the in vitro method of the present invention, culturing said luminal progenitor (LP) cells in a collagen gel is carried out in a culture medium for at least 14 days. The collagen gel used in step (ii) of the in vitro method of the present invention may be composed of one collagen type or a mixture of collagen types. A collagen type is, for example, type I, II, III, IV or V, with the type I being preferred. The collagen concentration may be in the range of about 0.5 mg/ml to about 3 mg/ml, preferably of about 0.8 mg/ml to about 2.0 mg/ml, more preferably of about 1.0 mg/ml to about 1.5 mg/ml, and most preferably of about 1.3 mg/ml. The term comprises attached and free-floating collagen gels.

The term “attached gel” as used herein, refers to a rigid collagen gel that sticks to the surface of the cell culture dish. This is in contrast to a “floating gel” that has been mechanically detached from the cell culture dish after polymerization of the gel and is thereby able to float in the cell culture medium. A floating gel is therefore more compliant than an attached gel and can e.g. contract or expand.

In one preferred embodiment of the in vitro method of the present invention, the collagen gel can be a collagen-I gel that is attached or free-floating in growth medium.

Thus, in one embodiment, as ECM surrogate, the inventors of the present invention employed collagen type I gels that are either floating or attached. Typically, 3D models employ Matrigel as ECM surrogate, which resembles the basal lamina of the breast. However, during cancer cell invasion, the basal lamina is breached. Therefore, upon onset of invasion, cancer cells come in direct contact with collagen type I, the main structural component of the ECM. The inventors of the present invention have found that interactions between LP cells and the collagen gel as described herein are crucial for invasion and branched structure formation and the formation of a multicellular organoid unit that morphologically and/or functionally recapitulates invasive and/or ductal cell growth.

Moreover, if a different cell type is employed (e.g. basal mammary epithelial cells), outgrowing structures do not resemble e.g. NST carcinoma characteristics. In addition, LPs in collagen gels have previously not shown invasive capacity when cultured in other media or when employing another ECM surrogate as described herein, such as Matrigel.

In one specific embodiment, without being limited to it, the gel preparation process with e.g. freshly sorted LP cells may take around 1.5 h and may be followed by a medium exchange after about 5 days and consecutive media exchanges about every 2-3 days. The full growth process may take approximately 14 days with the main organoid invasion/elongation phase being e.g. between about day 5 and about day 10. Hereby, no differences between floating or attached collagen type I gels was observed. During this phase, compounds with suspected anti-invasive or anti-migratory effect as defined herein may be added. If they possess the suspected impact on the organoid, invasion/migration may discontinue, which can be observed easily via morphological read-out, which may be preferably analysis of information on cellular movements, cellular velocity, duct elongation or ECM remodeling acquired via life cell imaging or via microscopic analysis of fixated samples.

It is further preferred for the in vitro method of the present invention that the one or more growth factor(s) of the culture medium is a ligand of the ErbB receptor family, preferably the epidermal growth factor (EGF) or an analogue thereof.

The ErbB receptor family means a family of proteins containing four receptor tyrosine kinases, structurally related to the epidermal growth factor receptor (EGFR), its first discovered member. In humans, the family includes Her1 (EGFR, ErbB1), Her2 (Neu, ErbB2), Her3 (ErbB3), and Her4 (ErbB4). The gene symbol, ErbB, is derived from the name of a viral oncogene to which these receptors are homologous: erythroblastic leukemia viral oncogene. Insufficient ErbB signaling in humans is associated with the development of neurodegenerative diseases, such as multiple sclerosis and Alzheimer's Disease, while excessive ErbB signaling is associated with the development of a wide variety of types of solid tumor.

All four ErbB receptor family members have a similar structure. This structure is made up of an extracellular region or ectodomain or ligand binding region that contains approximately 620 amino acids, a single transmembrane-spanning region containing approximately 23 residues, and an intracellular cytoplasmic tyrosine kinase domain containing up to approximately 540 residues. The extracellular region of each family member is made up of 4 subdomains, L1, CR1, L2, and CR2, where “L” signifies a leucine-rich repeat domain and “CR” a cysteine-rich region, and these CR domains contain disulfide modules in their structure (8 disulfide modules in the CR1 domain and 7 modules in the CR2 domain). These subdomains are also referred to as domains I-IV, respectively. The intracellular/cytoplasmic region of the ErbB receptor consists mainly of three subdomains: A juxtamembrane with approximately 40 residues, a kinase domain containing approximately 260 residues and a C-terminal domain of 220-350 amino acid residues that become activated via phosphorylation of its tyrosine residues that mediates interactions of other ErbB proteins and downstream signaling molecules.

The term “ligand of the ErbB receptor family” means in the context of the present invention a compound that is able to bind to any of the above-described members of the ErbB receptor family.

It is preferred for the in vitro method of the present invention that the one or more growth factor(s) of the culture medium is the epidermal growth factor (EGF).

It is also preferred for the in vitro method of the present invention that the one or more growth factor(s) of the culture medium is/are (an) analogue(s) of the epidermal growth factor (EGF), which may be transforming growth factor-alpha (TGFA), heparin-binding EGF-like growth factor (HBEGF), betacellulin (BTC), amphiregulin (AREG), epiregulin (EREG) or epigen (EPGN).

The in vitro method of the present invention further comprises that in one preferred embodiment the one or more inhibitor(s) of cell contractility of the culture medium is a Rho-kinase (ROCK) inhibitor, preferably Y-27632 or thiazovivin, or a myosin inhibitor, preferably para-amino blebbistatin.

In one preferred embodiment of the in vitro method of the present invention the one or more inhibitor(s) of cell contractility of the culture medium is a Rho-kinase (ROCK) inhibitor. Thus, the present inventors discovered that addition of a ROCK inhibitor can increase formation of a multicellular organoid unit that morphologically and/or functionally recapitulates invasive and/or ductal cell growth. Thus, a ROCK inhibitor can be added to improve cell culture conditions.

A “ROCK inhibitor” as used herein is a compound that acts as an inhibitor of Rho-associated protein kinase, i.e. reduces or even abolishes ROCK functionality. The capability of a compound to act as a ROCK inhibitor can be assessed by various means, e.g. by determining its ability to compete with ATP for binding to ROCK and/or by assessing its effects on cell morphology, G1-S transition and cytokinesis as described in Ishizaki T. Mol. Pharmacol. 2000 May; 57 (5): 976-83. The inhibitor may be either unspecific or specific for either of the ROCK isoforms ROCK1 and/or ROCK2. ROCK inhibitors known in the art have been reviewed in Liao et al. J. Cardiovasc. Pharmacol. 2007 July; 50 (1): 17-24 and include, for example, fasudil, Y-27632, thiazovivin, Y39983, Wf-536, SLx-2119, azabenzimidazole-aminofurazans, DE-104, olefins, isoquinolines, indazoles, pyridinealkene derivatives, H-1152P, ROKα inhibitor, XD-4000, 4-(1-aminoalkyl)-N-(4-pyridyl)cyclohexane-carboxamides, HMN-1152, rhostatin, BA-210, BA-207, BA-215, BA-285, BA-1037, Ki-23095, VAS-012, with Y-27632 or thiazovivin are particularly envisaged for use in the in vitro method, the method of screening or the culture medium of the present invention.

For the in vitro method of the present invention it is even more preferred that the one or more inhibitor(s) of cell contractility of the culture medium is Y-27632 or thiazovivin as ROCK-inhibitor. The present inventors have observed that a culture medium comprising Y-27632 or thiazovivin as a ROCK inhibitor is one being particularly useful for the methods of the present invention.

In one preferred embodiment, the one or more inhibitor(s) of cell contractility of the culture medium is a Rho-kinase (ROCK) inhibitor, preferably Y-27632 or thiazovivin, and is comprised in the culture medium in a concentration of about 1-10 UM, preferably of about 2-8 μM, more preferably of about 4-6 μM, and even more preferably of about 5 μM.

In one preferred embodiment, the one or more inhibitor(s) of cell contractility of the culture medium is a myosin inhibitor, preferably para-amino blebbistatin. In one more preferred embodiment, the one or more inhibitor(s) of cell contractility of the culture medium is a myosin inhibitor, preferably para-amino blebbistatin, and is comprised in the culture medium in a concentration of about 1-20 μM, preferably of about 5-15 μM, more preferably of about 8-12 μM, and even more preferably of about 10 μM.

In one preferred embodiment of the in vitro method of the present invention, the culture medium further comprises one or more selected from the group consisting of N-acetylcysteine, neuregulin 1, a vitamin, preferably nicotinamide, an antibiotic, a fibroblast growth factor (FGF), preferably FGF7 or FGF10, a MAP kinase inhibitor, preferably SB202190, a supplement and a buffer. In one preferred embodiment of the in vitro method of the present invention, the culture medium may further comprise N-acetylcysteine. In one preferred embodiment of the in vitro method of the present invention, the culture medium may further comprise neuregulin 1. In one preferred embodiment of the in vitro method of the present invention, the culture medium may further comprise, a vitamin, preferably nicotinamide. In one preferred embodiment of the in vitro method of the present invention, the culture medium may further comprise an antibiotic, preferably penicillin or streptomycin. In one preferred embodiment of the in vitro method of the present invention, the culture medium may further comprise a fibroblast growth factor (FGF), preferably FGF7 or FGF10. In one preferred embodiment of the in vitro method of the present invention, the culture medium may further comprise a MAP kinase inhibitor, preferably SB202190. In one preferred embodiment of the in vitro method of the present invention, the culture medium may further comprise a supplement, for example B27 50× or GlutaMax 100×. In one preferred embodiment of the in vitro method of the present invention, the culture medium may further comprise a buffer, more preferably Hepes buffer.

In one further preferred embodiment of the in vitro method of the present invention, the provided luminal progenitor (LP) cells are genetically modified, preferably one or more gene(s) are knocked-out in the provided luminal progenitor (LP) cells. For example, if E-cadherin is knocked out or E-cadherin is blocked, this resulted in diffusely invading organoids and a morphology that is similar to ILC, a subtype of cancer in which E-cadherin is usually lost. Invasive growth occurs in both cases of IDC and ILC, however, IDC and ILC are two morphologically different types of invasive breast cancer and said knock-out enables to morphologically recapitulate especially ILC.

In one preferred embodiment of the in vitro method of the present invention, the multicellular organoid unit morphologically and/or functionally recapitulates low-grade invasive processes of mammary cancer, preferably of low-grade carcinoma of no special type (NST). Low grade invasive processes of mammary cancer are defined and characterized by a high degree of well-differentiated ductal network formation. Carcinoma of NST is synonymous to invasive ductal carcinoma and can be detected by absence of a basal cell layer and expression of the luminal markers CK8/18, GATA-3, ZO-1 and mucin-1.

Thus, according to the method of the present invention, LPs may be seeded into collagen gels, resembling the extracellular matrix in the mammary gland. Following this, the single cells invade the matrix, while forming a clonal branched ductal network. The thereby arising branched structures may possess hallmarks of low-grade invasive cancer formation, such as lumen formation and luminal lineage marker expression. Especially, in one embodiment, the multicellular organoid unit may be similar to the morphology of invasive carcinomas of no special type (NST). The correlation of these organoids to invasive carcinomas is further corroborated by cancer associated mechanisms exploited during invasion such as matrix degradation and fiber alignment.

In step (iii) of the in vitro method of the present invention, it is determined whether a multicellular organoid unit has been formed in step (ii). In one embodiment, it is preferred for said in vitro method that determining whether a multicellular organoid unit is formed is by determining whether an invasive organoid or a ductal structure or one or more branch-point(s) is/are comprised in said multicellular organoid unit. It is preferred in this connection that “one or more branch point(s)” is at least two branch-points.

A “multicellular organoid unit” according to the present invention and as used in the context of the present invention is a multicellular structure that may be formed by at least a single cell. It is in particular envisaged that the at least one single cell is a luminal progenitor (LP) cell as described herein. The multicellular organoid unit morphologically and/or functionally recapitulates invasive and/or ductal cell growth as defined herein above. The term “multicellular organoid unit” may also comprise spheres, sticks and branched structures. Spheres are round and non-invasive. Sticks are ductal and invasive structures. Sticks and branched structures only differ in that branched structures have at least two points at which the structures branch, which then comes closer to the morphology of cancer as known from histopathology.

However, though a multicellular organoid unit is ideally morphologically and/or functionally identical to invasive and/or ductal cell growth, preferably mammary luminal cell invasion, it cannot be excluded that there may be differences. These differences are reflected in the term “organoid”, meaning it is an organ structure (i.e. an entire organ or functional part thereof) that is formed and grown ex vivo, which ideally morphologically and/or functionally resembles an organ structure. The same is true for the term “resemble” or “recapitulate”, which can be used interchangeable herein. It means that a multicellular organoid unit is/behaves like an organ structure and thus morphologically and/or functionally behaves like a (natural) organ structure. However, in contrast to a (natural or in vivo) organ, an organoid structure is formed and grown ex vivo. However, nonetheless, a multicellular organoid unit shares identity with the invasive and/or ductal cell growth, preferably with low-grade invasive processes of mammary cancer, more preferably of low-grade carcinoma of no special type (NST), as regards morphology in that it comprises e.g. ductal structures and multiple branch-points. From a functional perspective, a multicellular organoid unit is capable of contraction. Contraction may be tested as described herein.

A multicellular organoid unit according to the present invention is preferably considered to morphologically and/or functionally recapitulate low-grade invasive processes of mammary cancer, more preferably of low-grade carcinoma of no special type (NST), when it comprises ductal structures and/or multiple branch-points. It may also comprise alveoli at the tip of the ducts. Presence of the aforementioned features in a multicellular organoid unit can be easily assessed by the skilled person using visual examination, e.g. bright-field microscopy.

In one embodiment, it is preferred for said in vitro method of the present invention that the multicellular organoid unit is a multicellular breast organoid unit.

It is further envisaged that the multicellular organoid unit is responsive to hormones and/or growth factors. Hormones include e.g. steroid hormones, like estrogen, progesterone and androgens, pituitary hormones like prolactin, human growth hormone, and other peptide hormones like gluco- and mineralcorticoids, as well as insulin.

The term “one or more growth factors” according to the present invention may include the following families: EGF (Epidermal Growth Factors), IGF (Insulin-like growth Factors), FGF (Fibroblast Growth Factors), Wnt (Wingless), TGF-beta (Transforming Growth Factor beta), Notch, shh (sonic hedgehog). Included are endogenous and recombinant factors, precursors and derivatives, as well as endogenous, recombinant and synthetic agonists and antagonists. Responsiveness to hormones and growth factors renders the multicellular unit of the present invention a suitable substrate to test compounds for their ability to elicit a physiologically response.

The method of the invention may further comprise a step of determining whether the obtained multicellular organoid unit is capable of contracting a collagen gel.

Contraction of the collagen gel may be quantified by measurement of the gel size at various times with a ruler or with image analysis software, such as NIH Image or Image Pro-Plus (MediaCybernetics) and can be correlated to breast stem cell content.

The present invention further relates to a method of screening for an anti-migratory drug, comprising the following steps:
a) Bringing a multicellular organoid unit obtained by the in vitro method according to the present invention into contact with a compound suspected of being an anti-migratory drug, and
b) determining whether or not said compound elicits a cellular response in the multicellular organoid unit, with the proviso that when said compound elicits a cellular response compared to a reference state, said compound is an anti-migratory drug.

A “cellular response” can be e.g. according to the present invention cell elongation arrest, cell proliferation arrest, growth arrest, apoptosis, necrosis, DNA damage, inhibition of differentiation, migration arrest, and changes in the morphology of cells, preferably cell elongation arrest. Thus, in one preferred embodiment of the method of screening of the present invention, the cellular response is selected from the group consisting of cell elongation arrest, cell proliferation arrest, growth arrest, apoptosis, necrosis, DNA damage, inhibition of differentiation, migration arrest, and changes in the morphology of cells, more preferably cell elongation arrest. The “reference state” of the method of screening of the present invention is the state or condition before or without applying the compound suspected of being the anti-migratory drug.

Cellular responses can be assessed using standard protocols known in the art. Compounds that can be tested for their ability to provoke a cellular response include a drug, hormone, growth factor, antibody, nucleotide molecule, peptide, protein or (co-cultured) cell.

The term “anti-migratory” as used herein means inhibiting movement of cells in particular directions to specific locations. Said term, when used herein also comprises “anti-migratory” and “anti-invasive”.

As used in the context of the present invention, the term “anti-migratory” means inhibiting the spread of a disease-producing agency, such as cancer cells, from the initial or primary site of disease to another part of the body.

The term “anti-invasive” as used herein means inhibiting the tendency to spread, especially in a quick or aggressive manner, e.g. of cancer cells tending to infiltrate surrounding healthy tissue.

The present invention also relates to a culture medium comprising:
one or more growth factor(s),
one or more inhibitor(s) of cell contractility,
one or more serum component or serum substitute, and
further one or more of the group consisting of N-acetylcysteine, neuregulin 1, a vitamin, preferably nicotinamide, an antibiotic, a fibroblast growth factor (FGF), preferably FGF7 or FGF10, a MAP kinase inhibitor, preferably SB202190, a supplement and a buffer.

In one preferred embodiment of the culture medium according to the present invention, the one or more growth factor(s) is a ligand of the ErbB receptor family, preferably the epidermal growth factor (EGF) or an analogue thereof as defined herein above. In one more preferred embodiment of the culture medium according to the present invention, the one or more growth factor is EGF, even more preferably EGF in a concentration in the range of about 1 ng/ml to about 20 ng/ml, even more preferably EGF in a concentration in the range of about 2 ng/ml to about 10 ng/ml, and even more preferably EGF with a concentration of about 5 ng/ml.

In one preferred embodiment of the culture medium according to the present invention, the one or more inhibitor(s) of cell contractility is a Rho-kinase (ROCK) inhibitor, preferably Y-27632 or thiazovivin, or a myosin inhibitor, preferably para-amino blebbistatin, as defined herein above. In one preferred embodiment of the culture medium according to the present invention, the one or more inhibitor(s) of cell contractility is a Rho-kinase (ROCK) inhibitor. In one more preferred embodiment of the culture medium according to the present invention, the one or more inhibitor(s) of cell contractility is Y-27632 as Rho-kinase (ROCK) inhibitor. In one also more preferred embodiment of the culture medium according to the present invention, the one or more inhibitor(s) of cell contractility is thiazovivin as Rho-kinase (ROCK) inhibitor. In one preferred embodiment of the culture medium according to the present invention, the one or more inhibitor(s) of cell contractility is a myosin inhibitor. In one more preferred embodiment of the culture medium according to the present invention, the one or more inhibitor(s) of cell contractility is para-amino blebbistatin as myosin inhibitor. It is preferred for the culture medium according to the present invention, that the one or more inhibitor(s) of cell contractility is contained in a concentration in a range of about 1 μM to about 50 μM, even more preferably in a concentration in a range of about 2 μM to about 20 μM, and even more preferably with a concentration of about 5 μM.

In one preferred embodiment of the culture medium according to the present invention, the serum component or serum substitute is fetal calf serum (FCS). In one more preferred embodiment of the culture medium according to the present invention, the serum component or serum substitute is fetal calf serum (FCS). In one more preferred embodiment, the FCS is contained in the culture medium in a concentration in the range from about 0.1% to about 20%, even more preferred in a concentration in the range from about 0.2% to about 10%, even more preferred in a concentration in the range from about 0.2% to about 8%, even more preferred in a concentration in the range from about 0.2% to about 5%, even more preferred in a concentration in the range from about 0.2% to about 2.5%, even more preferred in a concentration in the range from about 0.2% to about 2%, even more preferred in a concentration in the range from about 0.2% to about 1%, even more preferably with a concentration of about 0.5%.

Thus, the inventors have developed a culture medium that allows recapitulation of invasive branching morphogenesis as well as long-term propagation of LP cells. The medium may comprise FCS and shows invasive and branching morphogenesis behavior in LP cells. Therefore, said culture medium according to the present invention was termed branched luminal organoid medium (BLOM, see Examples).

In one preferred embodiment of the culture medium according to the present invention, the culture medium further comprises N-acetylcysteine, more preferably N-acetylcysteine with a concentration in the range of about 0.5 mM to about 2 mM, even more preferably with a concentration in a range of about 1 mM to about 1.5 mM, and even more preferably with a concentration of about 1.25 mM.

In one preferred embodiment of the culture medium according to the present invention, the culture medium further comprises neuregulin 1, more preferably neuregulin 1 with a concentration in the range of about 2 nM to about 10 nM, even more preferably with a concentration in a range of about 3 nM to about 8 nM, and even more preferably with a concentration of about 5 nM.

In one preferred embodiment of the culture medium according to the present invention, the culture medium further comprises a vitamin, more preferably nicotinamide. More preferably, the vitamin, more preferably nicotinamide, is contained in the culture medium according to the present invention with a concentration in the range of about 1 mM to about 20 mM, even more preferably with a concentration in the range of about 2 mM to about 10 mM, and even more preferably with a concentration of about 5 mM.

In one preferred embodiment of the culture medium according to the present invention, the culture medium comprises additionally an antibiotic, more preferably penicillin or streptomycin. Even more preferably, the antibiotic, more preferably penicillin or streptomycin, is contained in the culture medium according to the present invention with a concentration in the range of about 10 μg/ml to about 500 μg/ml, even more preferably with a concentration in the range of about 20 μg/ml to about 200 μg/ml, and even more preferably with a concentration of about 100 g/ml.

In one preferred embodiment of the culture medium according to the present invention, the culture medium further comprises a fibroblast growth factor (FGF), preferably FGF7 or FGF10. Even more preferably, the fibroblast growth factor (FGF), more preferably FGF7 or FGF10, is contained in the culture medium according to the present invention with a concentration in the range of about 1 ng/ml to about 100 ng/ml, even more preferably with a concentration in the range of about 2 ng/ml to about 50 ng/ml, and even more preferably with a concentration in the range of about 5 ng/ml to about 20 ng/ml.

In one preferred embodiment of the culture medium according to the present invention, the culture medium further comprises a MAP kinase inhibitor, preferably SB202190. Even more preferably, the MAP kinase inhibitor, more preferably SB202190, is contained in the culture medium according to the present invention with a concentration in the range of about 100 nM to about 1000 nM, even more preferably with a concentration in the range of about 200 nM to about 800 nM, and even more preferably with a concentration in the range of about 400 nM to about 600 nM.

In a preferred embodiment of the culture medium according to the present invention, the culture medium may further comprise a supplement, such as GlutaMax 100× or B27 50×.

In a preferred embodiment of the culture medium according to the present invention, the culture medium may further comprise a buffer, such as Hepes. More preferably, the buffer is contained in the culture medium according to the present invention with a concentration in the range of about 1 mM to about 50 mM, even more preferably with a concentration in the range of about 2 mM to about 20 nM, and even more preferably with a concentration of about 10 mM.

In one embodiment of the culture medium according to the present invention, the culture medium may further comprise R-Spondin 3.

In one embodiment of the culture medium according to the present invention, the culture medium may further comprise Noggin.

In one embodiment of the culture medium according to the present invention, the culture medium may further comprise A-83-01.

In one embodiment of the culture medium according to the present invention, the culture medium may further comprise a basal medium, such as Advanced DMEM/F12.

Further, the present invention relates to a composition comprising those generated cells according to the present invention capable of differentiating to a multicellular organoid unit that morphologically and/or functionally recapitulates invasive and/or ductal cell growth or to a composition comprising the multicellular organoid unit as disclosed herein.

Said composition can be a pharmaceutical composition. The term “pharmaceutical composition” particularly refers to a composition suitable for administering to a human or animal, i.e., a composition containing components, which are pharmaceutically acceptable. In particular, a pharmaceutical composition comprises a luminal progenitor cell or a multicellular organoid unit as described herein together with a carrier, diluent or pharmaceutical excipient such as a buffer, a preservative and a tonicity modifier. Pharmaceutical compositions of the invention comprise a therapeutically effective amount of a luminal progenitor cell or a multicellular organoid unit and can be formulated in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form, which is particularly suitable for topical or oral administration.

The pharmaceutical composition may further comprise a solvent such as water, a buffer for adjusting and maintaining the pH value, and optionally further agents for stabilizing the luminal progenitor cell or multicellular organoid unit or agents for preventing degradation of the same. It may additionally comprise further luminal progenitor cells or multicellular organoid units and other pharmaceutically active agents, such as adjuvants etc.

“Therapeutically effective amount” means an amount of luminal progenitor cells or multicellular organoid units that elicit the desired therapeutic effect. The exact amount or dose depends on the purpose of the treatment, and will be ascertainable by one skilled in the art using known techniques. As is known in the art and described above, adjustments for age, body weight, general health, sex, diet, drug interaction and the severity of the condition may be necessary, and will be ascertainable with routine experimentation by those skilled in the art.

A variety of routes are applicable for administration of the pharmaceutical composition, including, but not limited to, orally, topically, transdermally, subcutaneously, intravenously, intraperitoneally, intramuscularly or intraocularly. However, any other route may readily be chosen by the person skilled in the art, if desired.

Additionally, the present invention also relates to the use of a culture medium according to the present invention as described herein in any of said methods according to the present invention as described herein.

It must be noted that as used herein, the singular forms “a”, “an”, and “the”, include plural references unless the context clearly indicates otherwise. Thus, for example, reference to “the method” includes reference to equivalent steps and methods known to those of ordinary skill in the art that could be modified or substituted for the methods described herein.

All publications and patents cited in this disclosure are incorporated by reference in their entirety. To the extent the material incorporated by reference contradicts or is inconsistent with this specification, the specification will supersede any such material.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

When used herein, the term “about” is understood to mean that there can be variation in the respective value or range (such as pH, concentration, percentage, molarity, number of amino acids, time etc.) that can be up to 5%, up to 10% of the given value. For example, if a formulation comprises about 5 mg/ml of a compound, this is understood to mean that a formulation can have between 4.5 and 5.5 mg/ml. This term includes also the concrete number, e.g., about 5 mg/ml includes 5 mg/ml.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well-known in the art. Generally, nomenclatures used in connection with techniques of biochemistry, enzymology, molecular and cellular biology, microbiology, genetics and protein and nucleic acid chemistry and hybridization described herein are those well-known and commonly used in the art.

The methods and techniques of the present invention are generally performed according to conventional methods well-known in the art and as described in various general and more specific references that are cited and discussed throughout the present specification unless otherwise indicated. See, e. g., Sambrook et al., Molecular Cloning: A Laboratory Manual, 3rd ed., Cold Spring Harbor Laboratory Press, Cold Spring Harbor, N. Y. (2001); Ausubel et al., Current Protocols in Molecular Biology, J, Greene Publishing Associates (1992, and Supplements to 2002); Handbook of Biochemistry: Section A Proteins, Vol I 1976 CRC Press; Handbook of Biochemistry: Section A Proteins, Vol II 1976 CRC Press. The nomenclatures used in connection with, and the laboratory procedures and techniques of, molecular and cellular biology, protein biochemistry, enzymology and medicinal and pharmaceutical chemistry described herein are those well-known and commonly used in the art.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### EXAMPLES OF THE INVENTION

The following Examples illustrate the invention, but are not to be construed as limiting the scope of the invention.

### I. Experimental Procedures/Materials and Methods

### 1. Isolation and Culture of Human Mammary Epithelial Cells

Healthy mammary gland tissue was provided by the Nymphenburg Clinic for Plastic and Aesthetic Surgery and processed in accordance with the regulations of the ethics committee of the Ludwig-Maximilian University, Munich, Germany (proposal 19-989) as described previously^{35,63}. In brief, reduction mammoplasty tissue was minced with scalpels into 2-3 mm³ pieces. Next, tissue was digested with 300 U/mL collagenase I and 100 U/mL hyaluronidase and 1 μg/mL insulin for approximately 16 hrs. Finally, fragments were either cryopreserved immediately or a single cell suspension was made using trypsin-EDTA and dispase. If cells were cryopreserved as fragments, they were turned into a single cell suspension upon thawing using trypsin-EDTA and dispase. Prior to experiments, single cell suspensions were filtered through a 40 μm cell strainer. Age and parity of donors used in this study are catalogued in Table 1 given directly below.

TABLE 1
Reduction mammoplasty donors

| Donor | Age (years) | Parity |
|---|---|---|
| M16 | 17 | 0 |
| M28 | 38 | 1 |
| M35 | 48 | 0 |
| M36 | 63 | 3 |
| M42 | 41 | 0 |
| M44 | 19 | 0 |
| M45 | 54 | 1 |
| M46 | 51 | 1 |
| M47 | 55 | 2 |
| M48 | 49 | 1 |
| M50 | 58 | 1 |
| M51 | 37 | 2 |

Cells were grown in an incubator at 37° C. with 5% CO₂, oxygen levels were maintained at 3%. Neubauer chambers were used for cell counting and in order to verify singulation in single cell suspensions. A stock of BLOM-medium foundation containing N-acetylcysteine, nicotinamide, GlutaMax 100×, Hepes and penicillin/streptomycin (see also Table 1) was kept for up to 2 months at 4° C. The full medium was always prepared freshly and used within 24 hrs. Table 2 given below shows a full list of compounds.

TABLE 2
Media compositions (BCOM and BLOM) with respective concentrations

| BCOM | BLOM | Concentrations | | Catalog number | Supplier |
|---|---|---|---|---|---|
| N-Acetylcysteine | N-Acetylcysteine | 1.25 | mM | A9165 | Sigma |
| Nicolinamide | Nicolinamide | 5 | mM | N0636 | Sigma |
| GlutaMax 100× | GlutaMax 100x | 1× | | 35050061 | Life Technologies |
| Hepes | Hepes | 10 | mM | H4034 | Sigma |
| Penicillin/Streptomycin | Penicillin/Streptomycin | 100 | μg/ml | 5000956 | Invitrogen |
| Neuregulin 1 | Neuregulin 1 | 5 | nM | 100-03 | Pepro Tech |
| EGF | EGF | 5 | ng/ml | AF-100-15 | Pepro Tech |
| SB202190 | SB202190 | 500 | nM | S7067 | Sigma |
| B27 50x | B27 50× | 1× | | 17504044 | Life Technologies |
| Y-27632 | Y-27632 | 5 | μM | Y0110 | Biomol |
| R-Spondin 3 | R-Spondin 3 | 250 | ng/ml | 120-44 | Pepro Tech |
| FGF7 | FGF7 | 5 | ng/ml | 100-19 | Pepro Tech |
| FGF10 | FGF10 | 20 | ng/ml | 100-26 | Pepro Tech |
| Noggin | Noggin | 100 | ng/ml | 120-10C | Pepro Tech |
| A-83-01 | A-83-01 | 500 | nM | 2939 | Tocris |

| | | | | |
|---|---|---|---|---|
| | FCS | 0.5% | AC-SM-0160 | Anprotec |
| Advanced DMEM/F12 | Advanced DMEM/F12 | | 12634010 | Life Technologies |

### 2. Fluorescence Activated Cell Sorting (FACS)

For sorting of bulk primary cells, single cell suspensions in 0.1% BSA were stained with CD31-V450, CD45-V450, CD49f-PE, EpCAM-FITC and CD10-APC. This can be seen from Table 3 directly given below.

TABLE 3
Antibodies and cellular stains used for fluorescence activated cell sorting

| | | | Volume for staining | Catalog | |
|---|---|---|---|---|---|
| Epitope (Clone) | Conjugation | Host | 1 × 10\hat{ }6 cells | number | Supplier |
| 7-AAD | — | — | 4 | 559925 | BD |
| CD31 (WM59) | V450 | Mouse | 1 | 561653 | BD |
| CD45 (HI30) | V450 | Mouse | 1 | 560368 | BD |
| CD326/EpCAM | FITC | Mouse | 10 | GTX79849 | Biozol |
| (VU-D19) | | | | | |
| CD49f (GOH3) | PE | Rat | 5 | 555736 | BD |
| CD10 (HI10A) | APC | Mouse | 5 | BLD-312210 | Biozol |
| SYTOX ™ Blue | — | — | 1/1000* | S34857 | Life Technologies |

*Dilution based on sample volume

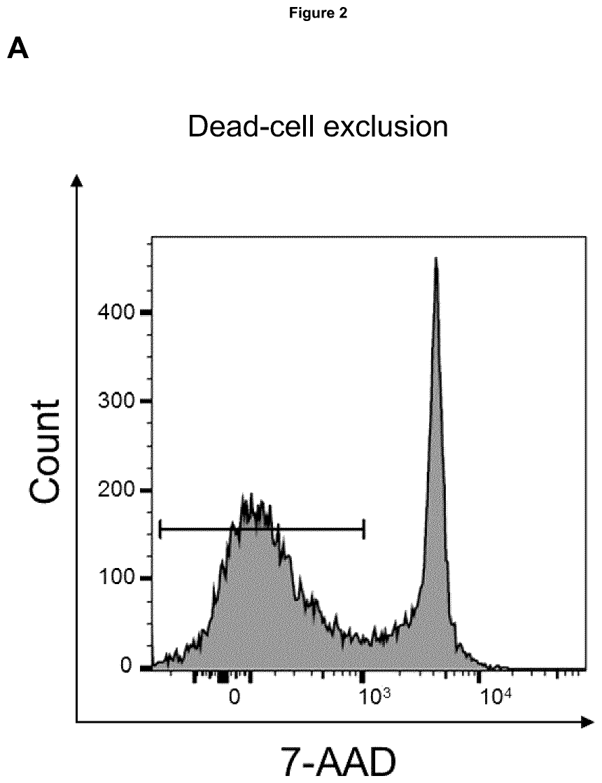

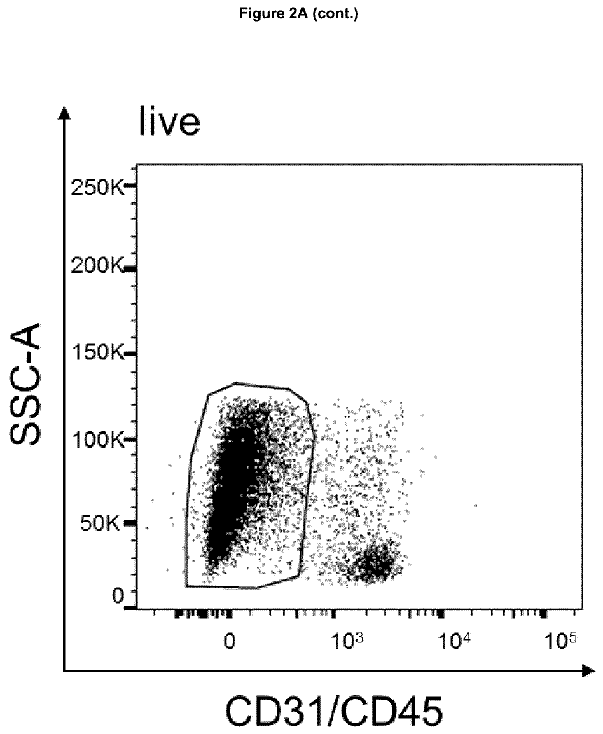

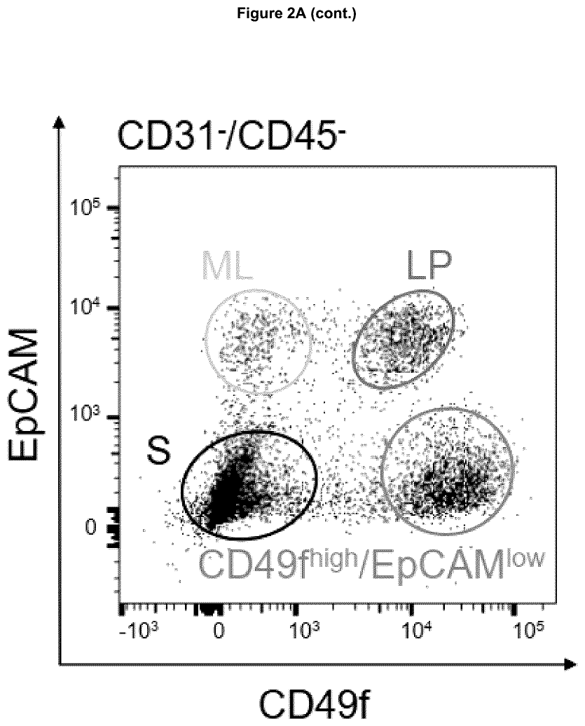

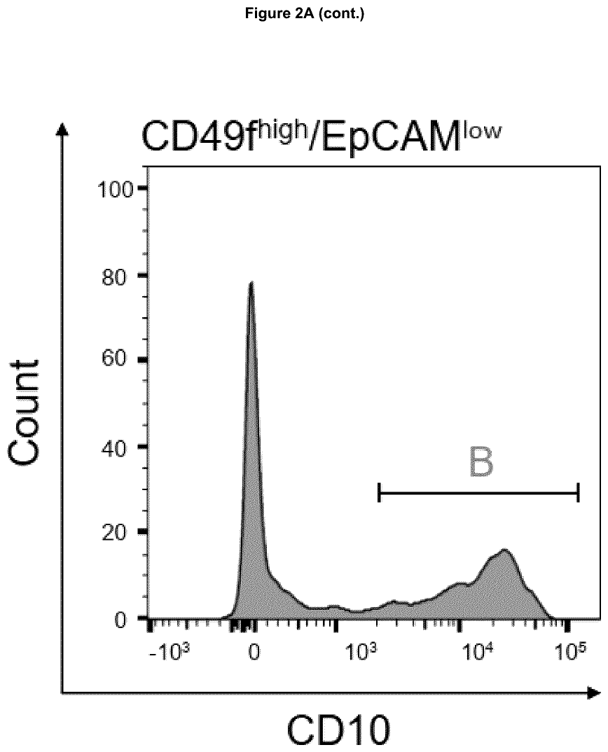

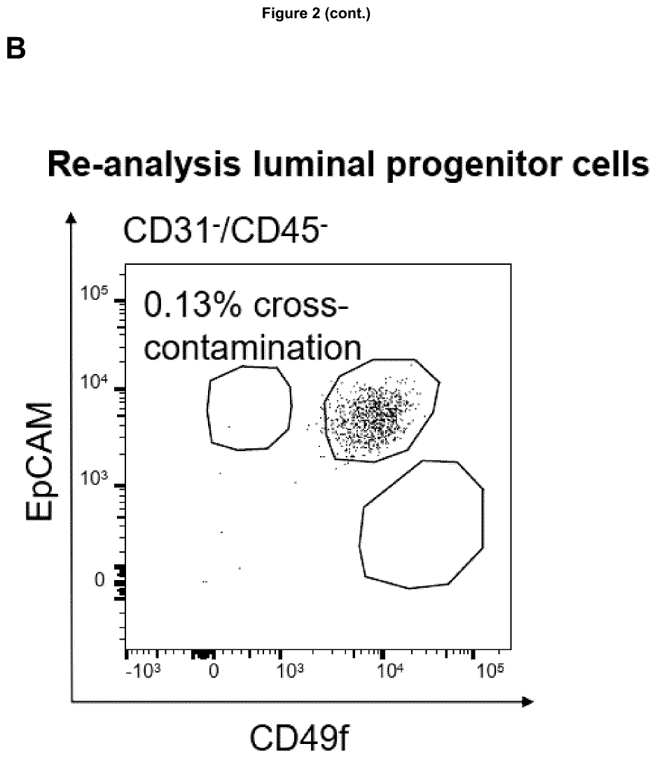

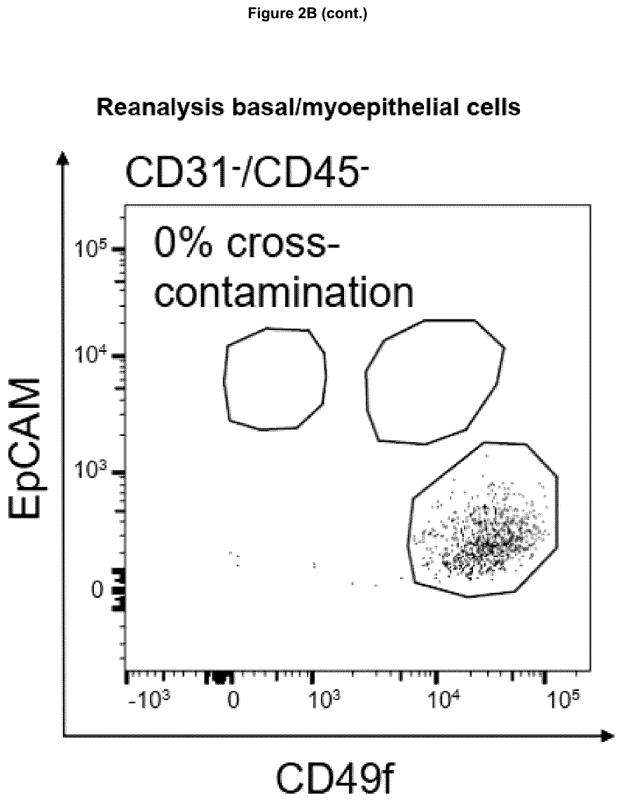

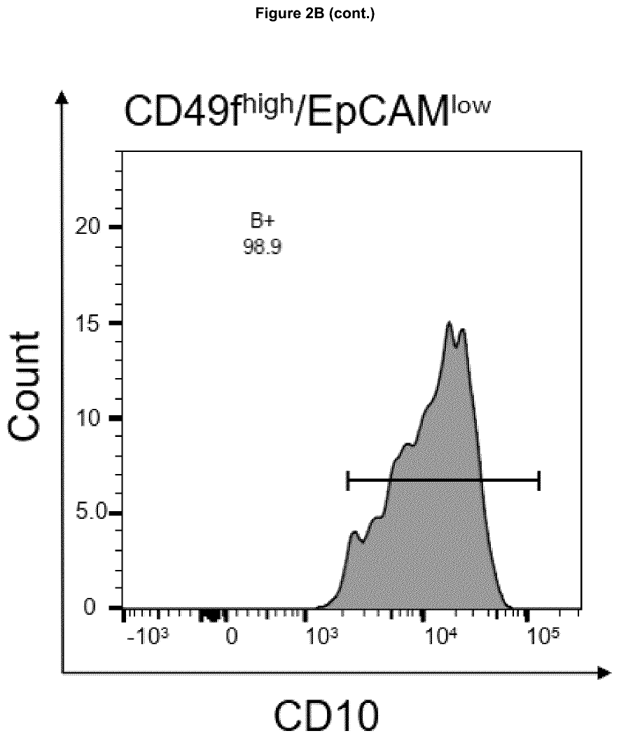

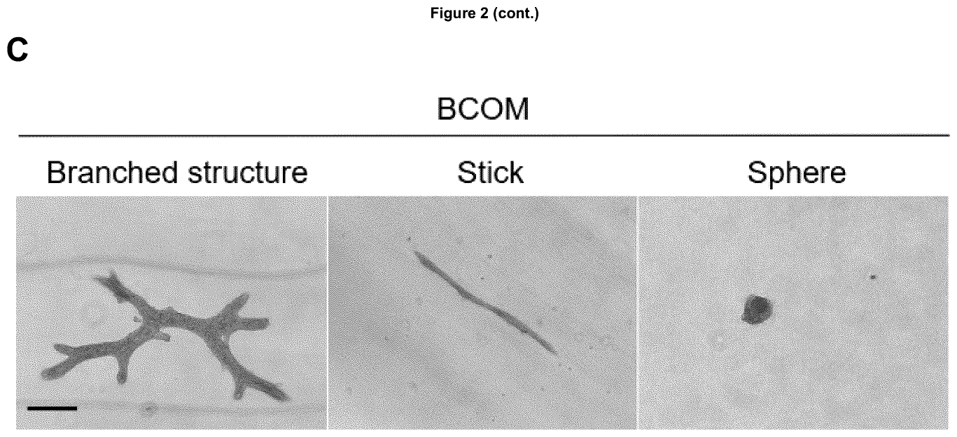

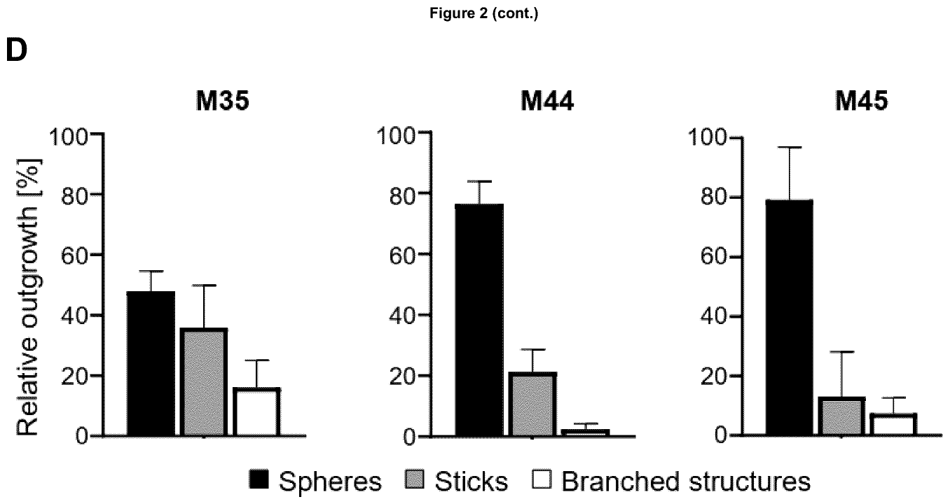

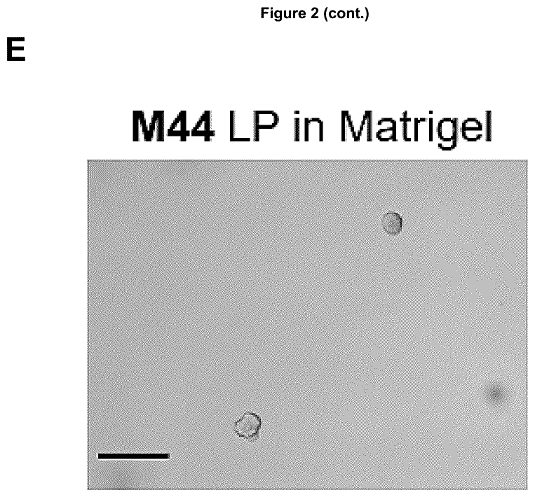

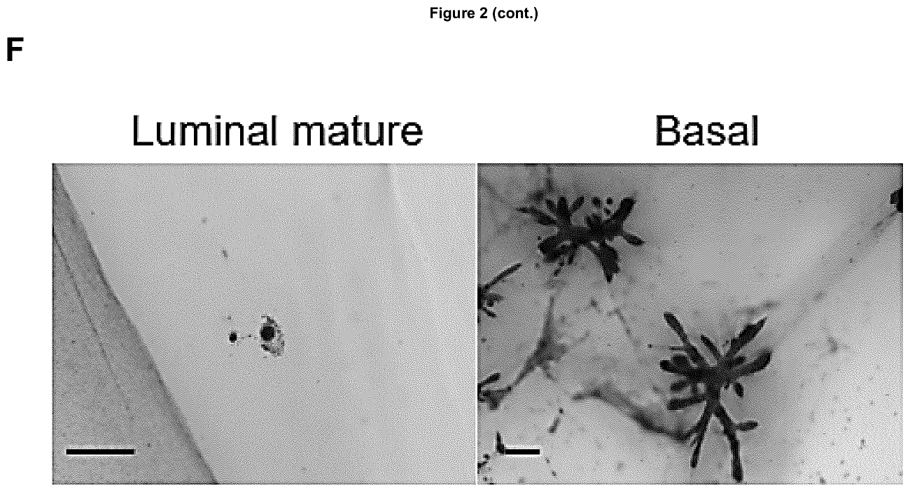

Prior to the sort, 7AAD (BD Biosciences, 559925) was added for dead cell exclusion. In case of sorting transfected GFP positive cells, SYTOX™ Blue (Life Technologies, S34857) was used for dead cell separation. Cells were sorted on a FACSAria™ III cell sorter (BD Biosciences) using the FACSDiva™ 6.0 Software. Side scatter (SSC-A) and Forward scatter (FSC-W) were used to exclude cell debris, further, cell doublets were excluded using both FSC-A/FSC-W and SSC-A/SSC-W. Sample purity of bulk sorts was determined by re-analyzing the sorted populations as shown in FIG. 2B. No re-analysis was performed for transfected cells. Further analysis of the data was performed with the FLOWJo_V10 Software (FlowJo LLC).

### 3. 3D Culture in Collagen I Gels

3D collagen type I gels were prepared as described previously^{35,63} with modifications. Single cell suspensions were mixed with BLOM foundation, rat tail collagen type I (Corning) and neutralizing solution. The final collagen concentration was 1.3 mg/ml. If the pH of the solution was <7.4, the pH was adjusted by addition of NaOH (1 M) up to a volume of 1 μl per 100 μl polymerization solution. After polymerization for 1 hr, 2× concentrated full medium was added onto the gels in a 1:1 volume ratio to the gel. Gels were encircled with a pipette tip for detachment. For cells that had not been in culture previously, the first medium exchange (from now on with 1× concentrated medium) was done after 5 d and then every 2-3 d. In case of cells that had been in 2D culture before, the first medium exchange was done between 3 d and 5 d. For all experiments except ELDA, per 1 ml of gel, between 250 and 2000 LP cells were seeded. In case of B+ and LM cells, between 2000 and 7500 cells were seeded per 1 ml of gel.

### 4. 3D Culture in Matrigel

A single cell suspension of primary luminal progenitor cells in BLOM was re-suspended in cold growth factor reduced Matrigel (Corning) on ice. 750 μL of this mixture was plated into a well of a 2-well μ-slide (Ibidi). After 30 min of polymerization at 37° C., 750 μL of BLOM was added.

### 5. Measurement of Structure Dimension and Assessment of Complexity

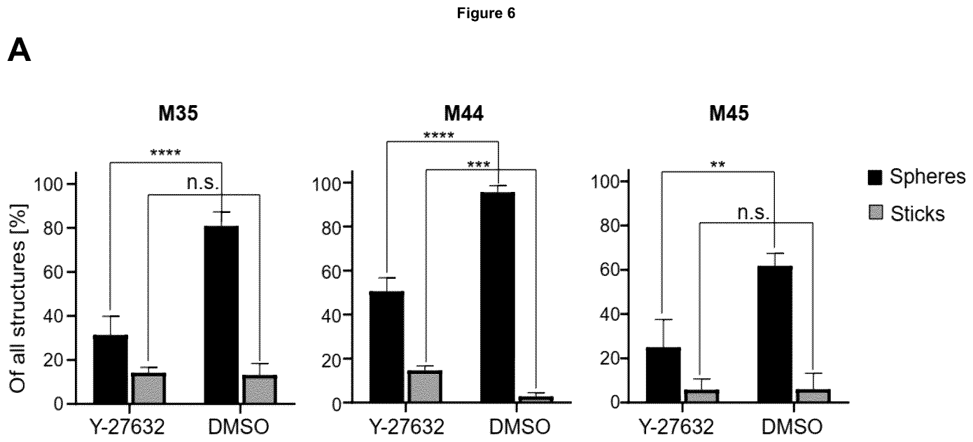

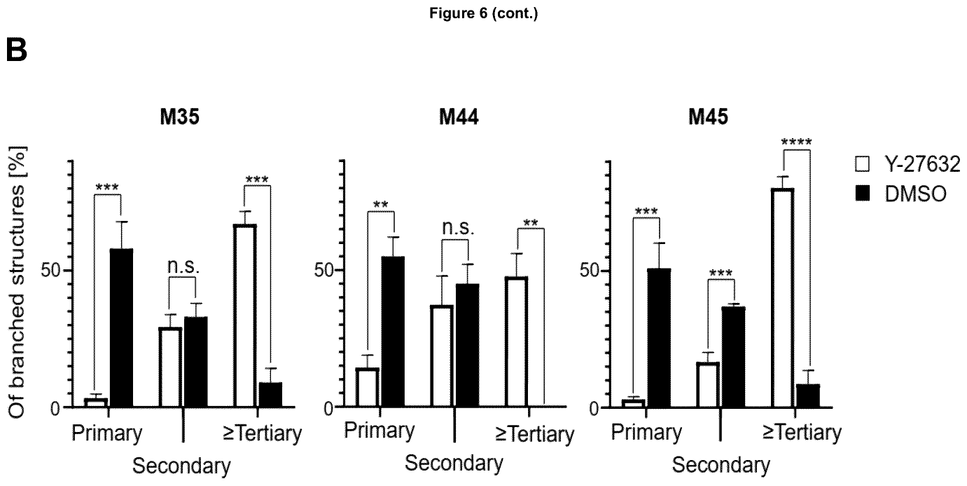

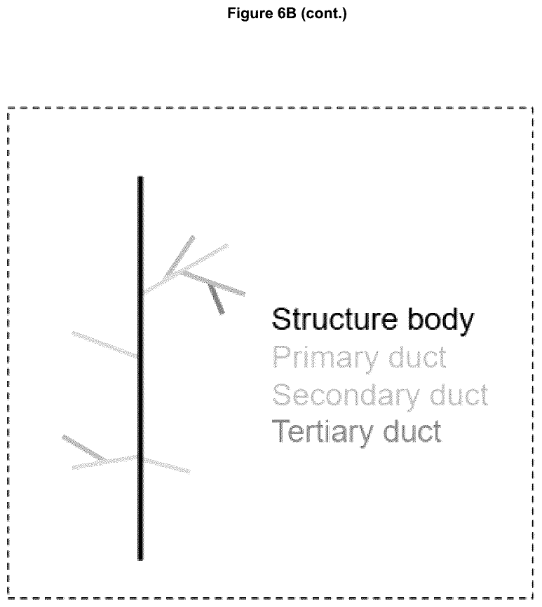

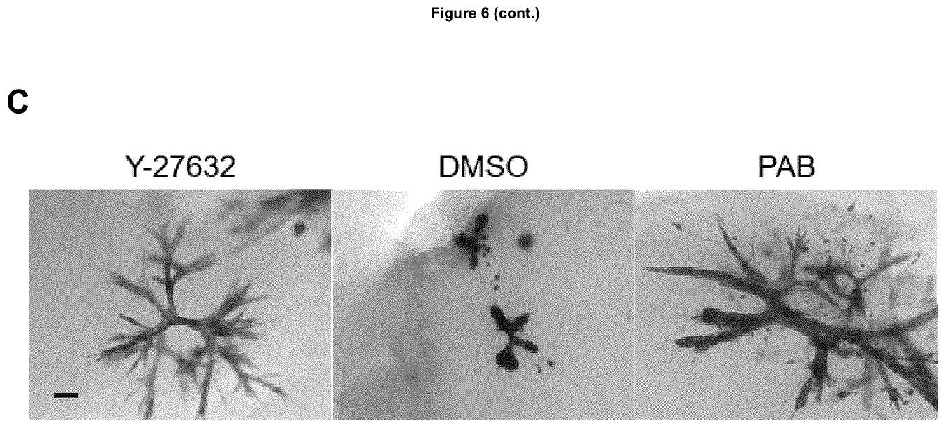

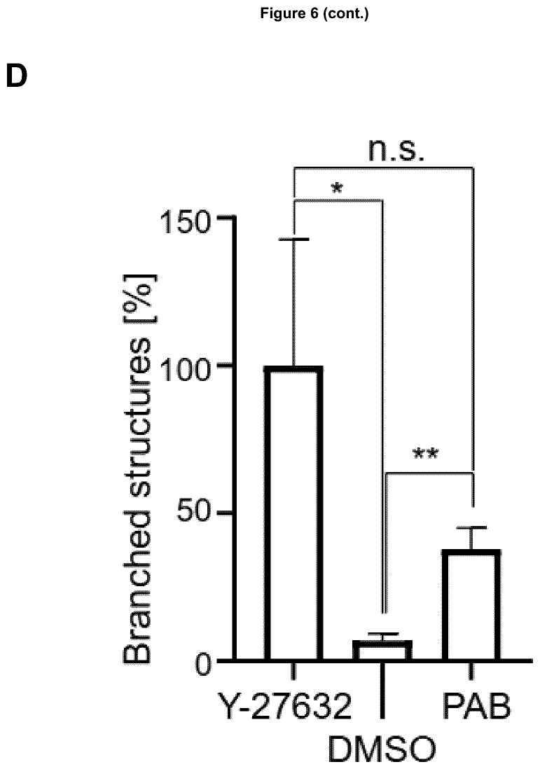

Images of carmine stained structures were evaluated with ImageJ. The size of an organoid was determined with the ImageJ area tool. Here, all tips of the structure were virtually connected with the ImageJ area tool and size was defined as the area covered by the organoid. For assessment of complexity, the longest branch was identified within each structure. The hierarchy of all other branches relative to this longest branch was classified (see FIG. 6B).

### 6. Extreme Limiting Dilution Analysis (ELDA)

The exact numbers of cells that were able to generate branched structures, the branched structure forming units (B-SFU) were determined by preparing gels in 6 different cellular densities. For each density, 6×24-well gels with a gel volume of 400 μl were prepared. Branched structures were defined by the presence of more than two branching points. Gels with at least one branched structure were counted as positive. Analysis was performed as described previously⁴⁵.

### 7. Carmine Staining

Carmine staining solution was prepared with a concentration of 0.5 g/l carmine and 1.15 g/l aluminium potassium sulfate in distilled water. For staining of 3D structures, gels were fixed with 4% paraformaldehyde for 15 min and incubated with carmine staining solution overnight. Images were acquired on a Leica DM IL LED microscope using a HiPlan 10×/0.22 PH1 objective. Three PBS washing steps were performed in between all steps described above.

### 8. Immunofluorescence

2D cells for immunofluorescence were cultured in 96-wells with optically clear bottom (PerkinElmer, 6005550). Immunofluorescence was performed as described previously^{35,63}. In brief, 2D cells or 3D cells (in collagen type I gels) were fixed with 4% paraformaldehyde for 15 min. Cells were permeabilized with 0.2% Triton X-100 for 2 min (2D) and 10 min (3D) respectively. Blocking was performed with 10% donkey serum in 0.1% BSA for 1 hr (2D) and overnight at 4° C. (3D) respectively. Primary antibodies in 0.1% BSA were incubated for 1 hr (2D) and overnight at 4° C. (3D) respectively. Respective dilutions are listed in Table 4 directly below.

TABLE 4
Primary antibodies used for immunofluorescence and blocking

| Epitope (Clone) | Conjugation | Host | Dilution | Catalog number | Supplier |
|---|---|---|---|---|---|
| CK8/18 (5D3) | — | Mouse | 1/250 | DLN-10750 | Dianova |
| GATA3 (L50-823) | — | Mouse | 1/250 | CM405 | Biocare Medical |
| ZO-1 (1A12) | — | Mouse | 1/100 | 339100 | Life Technologies |
| Mucin-1 | — | Rabbit | 1/100 | ab15481 | Abcam |
| Laminin | — | Rabbit | 1/100 | L9393 | Sigma |
| Phalloidin | Atto 647N | — | 1/250 | 65906 | Sigma |
| α-SMA | — | Rabbit | 1/100 | ab5694 | Abcam |
| p63 (EPR5701) | | Rabbit | 1/300 | ab124762 | Abcam |
| E-Cadherin | Alexa 488 | Rabbit | 1/50 | 3199 | CST |
| (24E10) | | | | | |
| E-Cadherin | — | Mouse | 1/25 | ab1416 | Abcam |
| (HECD1) | | | | | |
| Ki-67 | — | Rabbit | 1/300 | ab15580 | Abcam |

Secondary antibodies in 0.1% BSA were added for 2-3 hrs. Respective dilutions are listed in Table 5 directly given below.

TABLE 5
Secondary antibodies used for immunofluorescence

| Host/ | | Species | | Catalog | |
|---|---|---|---|---|---|
| Isotope | Conjugation | reactivity | Dilution | number | Supplier |
| Donkey/ | Alexa 488 | Mouse | 1/250 | A-21202 | Life |
| IgG | | | | | Technologies |
| Donkey/ | Alexa 594 | Rabbit | 1/250 | A-21207 | Life |
| IgG | | | | | Technologies |
| Donkey/ | Alexa 647 | Mouse | 1/250 | A-31571 | Life |
| IgG | | | | | Technologies |

Next, DAPI (167 ng/ml) was added for 2 min (2D) and 10 min (3D), respectively. Finally, Aqua-Poy/Mount mounting medium (Polysciences, 18606) was added to preserve staining. Three PBS washing steps were performed in between all steps described above. Images of cells grown in 2D were acquired on an Axio Imager. A M2m imaging microscope was used with a 40× objective. Images of cells grown in 3D were acquired on FLUOVIEW FV1000 inverted confocal laser scanning microscope equipped with four laser lines (405, 488, 543, and 633 nm) using UPLSAPO 60×, 40× and 20× objective lenses. Images were processed with Gimp 2.8.22 and ImageJ 1.52i.

### 9. Live-Cell Imaging

Live cell imaging was performed using a Leica SP8 lightning confocal microscope with an on-stage incubation system (Ibidi) regulating CO₂, O₂, humidity and temperature. Organoids were imaged during elongation phase between day 7 and 9 with a time separation of 10 min between each acquired image. For visualization, nuclei were labelled with 10 μM sirDNA (Spirochrome AG) 3 hours prior to the measurements. Accordingly, organoids were excited at 633 nm and the fluorescent signal was collected around 674 nm using a HCX PL APO 10×/0.40 CS dry objective. Bead displacements/deformation fields were visualized using ImageJ 1.48v. First, the fluorescence signal of the fluorescent nuclei at the first time step was summed up along the z-axis to visualize the shape of the organoid. Second the fluorescence channel of the beads was summed along the z-axis and subsequently summed up over all time steps. At last, both calculated pictures were merged into one image. Bead tracking was calculated using Matlab R2020b. First, the fluorescence channel of the beads was masked by an intensity threshold to reduce the background. Second, bead positions are defined by taking the center of an interpolated intensity grid. Third, individual bead tracks were calculated by matching bead coordinates in three consecutive images. Thereby, beads touching the boundaries or beads to close to each other were excluded to guarantee a correct tracking.

### 10. Collagen Labelling and Imaging

Atto 488 (Merck, 41051) was used to fluorescently label collagen type I as described previously⁸⁴. In brief, collagen was dialyzed at 4° C. to a pH of 7. Next, collagen was incubated overnight at 4° C. with Atto 488 to facilitate conjugation. Non-bound dye was removed by further dialysis for 8 hrs. Acid was added in a final dialysis overnight to prevent unwanted polymerization. The collagen network was visualized by confocal reflection and fluorescence microscopy using a Leica SP8 lightning confocal microscope. In particular, collagen gels were illuminated with 488 nm through a HC PL APO 40×/1.10 water immersion objective. Subsequently, the reflected light was collected at 488 nm and the emitted fluorescent light between 510 nm and 550 nm. The distance in between each slice was set to 1 μm. The degree of alignment was calculated as previously described⁶⁵.

### 11. Data Analysis

Analysis for structure prevalence, type, size and complexity was performed with GraphPad Prism 8. Nuclei velocity within the organoid was calculated via optical flow using the in Matlab implemented Farneback algorithm. By defining the branch axis, the velocities were separated in a parallel and orthogonal component. The directionality of nuclei migration was defined by calculating the ratio of parallel and orthogonal component. Consequently, at directionality equal to one parallel and orthogonal component are at the same order of magnitude. At higher or lower values, either parallel or orthogonal velocity component dominates. In order to visualize the migration patterns of individual cells, cells were manually tracked. Therefore, each nucleus was tracked three times and the resulting average path was calculated.

### 12. Inhibitor Treatment

Collagen degradation via MMPs was inhibited by using 10 μM Marimastat (Merck, 444289), which was added to the medium of elongating organoids prior to imaging. E-cadherin blocking was performed by addition of an E-cadherin blocking-antibody HECD1 in a dilution of 1:25 after 5 d of organoid culture.

### 13. Collagen Coating and 2D Culture of LP Cells

For 2D expansion of LPs, culture dishes were collagen type I coated. To this end, dishes were coated with rat tail collagen I in a concentration of 5 μg/cm² collagen I in PBS. Coating was performed for 2-3 hrs at 37° C. or overnight at 4° C. Notably, without collagen coating, freshly sorted LPs could not attach. During 2D culture, the medium as published by Sachs et al. 2018 was used. The first medium exchange was performed after 5 d, then every 1-2 d. For cell splitting, 0.15% Trypsin-EDTA was used which was neutralized with trypsin neutralizing solution (TNS). Cells were spun down, re-suspended in medium, counted and seeded again.

### 14. CRISPR-Cas9 Mediated Gene Knockout

The gRNAs were designed using benchling software. The following sgRNA sequences were used for targeting CDH1:

(SEQ ID NO: 1)
5′-ATAATAAAGACACCAACAGG-3′
and
(SEQ ID NO: 2)
5′-TTAGAAGCTTGTTGACACCG-3′.

String assembly gRNA cloning (STAgR) was used to clone the two gRNAs into a STAgR_Neo plasmid as described previously⁶⁶. Freshly sorted LP cells were expanded in 2D as described above. The cells were grown to confluency and then seeded in a density of 1.5×10\hat{ }4 cells/cm². After 6 hrs, the cells were transiently transfected with the TransIT-X2 transfection reagent (Mirus Bio LLC, MIR 6003) according to manufacturer's recommendation. Cas9-GFP expressing plasmid (pSpCas9 (BB)-2A-GFP) and the gRNA containing STAgR_Neo plasmid were co-transfected in a 1:3 molar ratio. Control transfections were performed with Cas9-GFP expressing plasmid and STAgR_Neo plasmid containing no gRNAs. Medium was exchanged 12-16 hrs after transfection. 48 hrs after splitting, cells were harvested for FACS. CRISPR/Cas9 induced deletions at the targeted locus were assessed after FACS sort for GFP-positive cells. To this end, DNA was isolated with DNAzol (Life technologies, 10503027) and PCR was performed with Q5 High Fidelity DNA Polymerase (NEB, M0491L) and primers flanking the target region, which are: 5′-GCTGTCTGGCTAGGTTGGAC-3′ (SEQ ID NO: 3) and 5′-GATCCAGCATGGGTTGACCA-3′ (SEQ ID NO: 4).

For assessment of editing efficiency on the protein level, gels were stained for E-cadherin and structures with and without E-cadherin were quantified. ImageJ was used for measurement of maximum structure width.

### 15. Immunohistochemistry on Tissue Sections

Three micrometer-thick paraffin sections were used for hematoxylin and eosin staining and consequent immunohistochemical stainings (IHC). IHC was performed using a fully automated slide preparation system (‘Benchmark XT System’; Ventana Medical Systems, Tucson, AZ, USA) using reagents and buffers from Ventana Medical Systems. Antibodies against p63 (clone: SFI-6, number: P1006C01, DCS, dilution: 1:50) GATA3 (clone: L50-823, number: CM405B, Biocare, dilution: 1:200) and E-cadherin (clone: EP700Y, number: 246R-16, Cell Marque, dilution: 1:100) were used.

### 16. Statistical Analysis

Box-and-whisker plots present data as median±25% and were generated with GraphPad Prism Software using the “min to max.” method. All other data are depicted as mean±standard deviation. All p values were calculated using an unpaired two tailed t-test for comparison of two groups. P>0.05 was considered not-significant (n.s.). P≤0.05 was considered significant. Significance is indicated as follows: *P≤0.05, **P≤0.01, ***P≤0.001, ****P≤0.0001.

### II. Results

### Example 1: Human Mammary Luminal Progenitor Cells Give Rise to Complex Branched Structures in Collagen Type I Gels

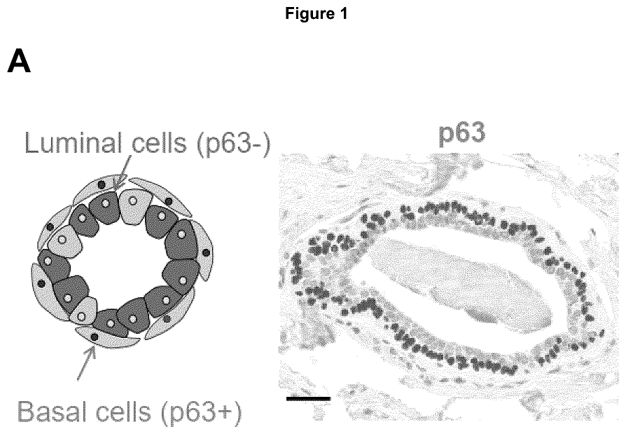

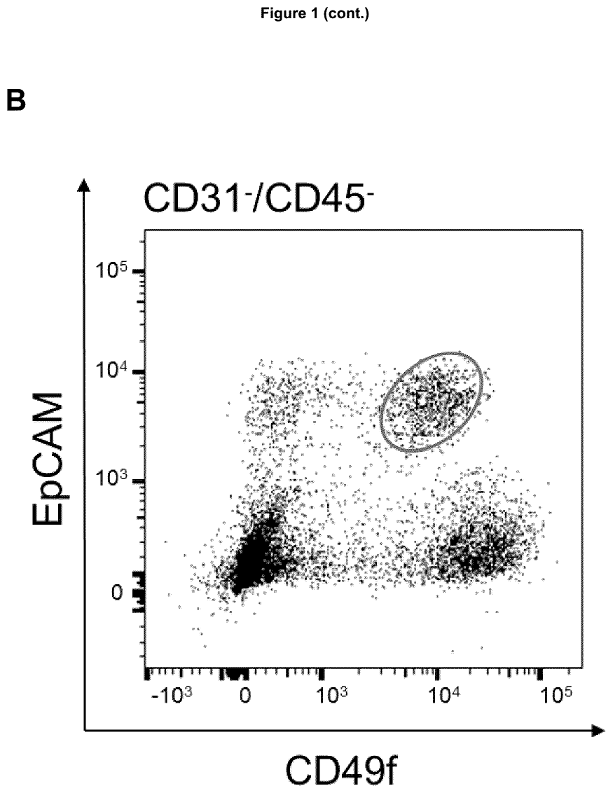

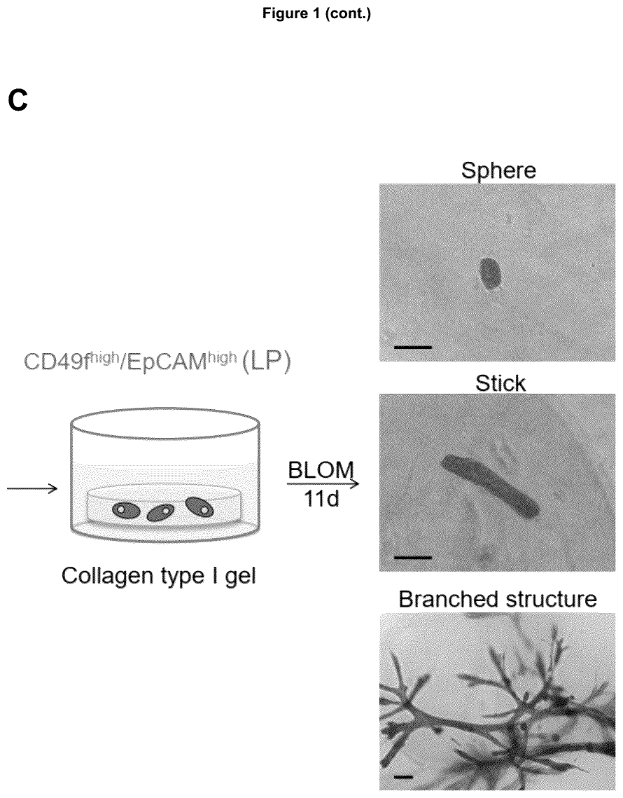

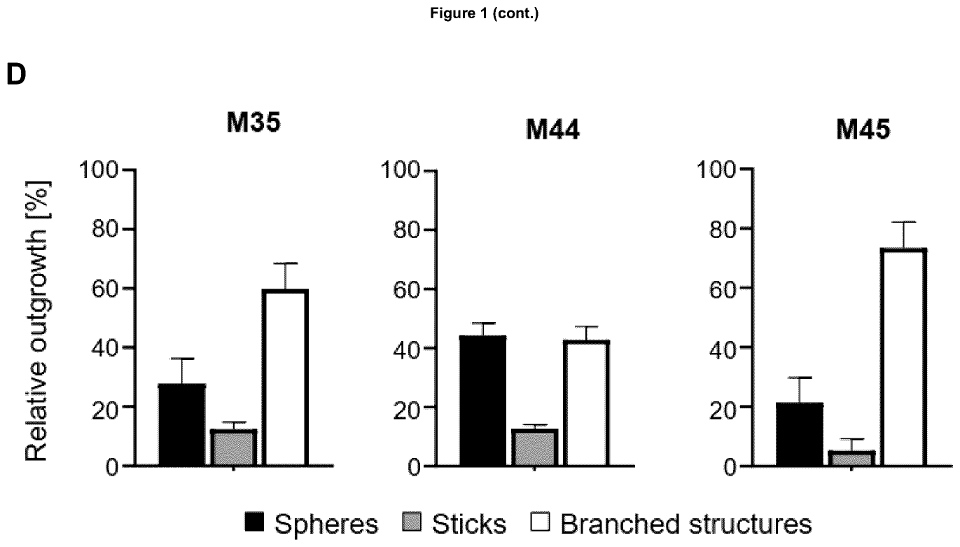

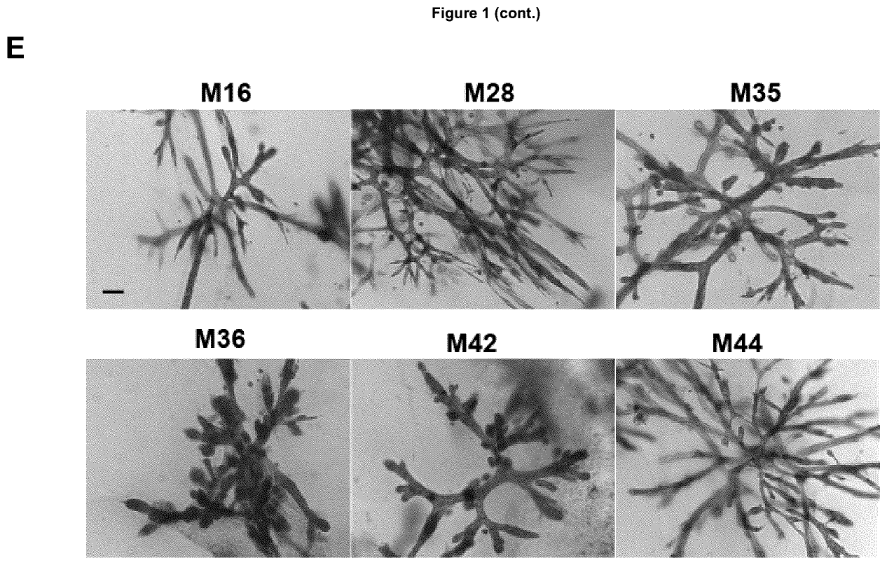

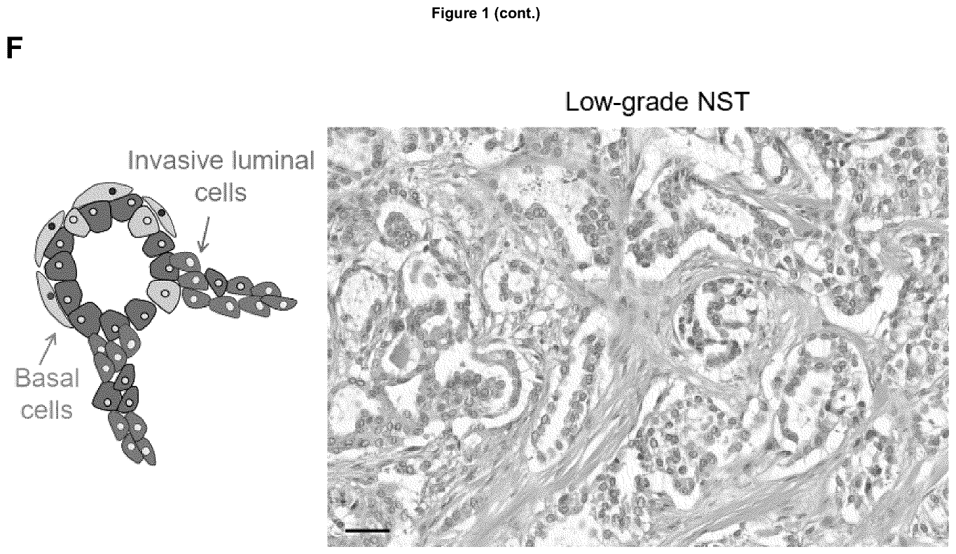

In order to recapitulate luminal cell invasion in vitro, the inventors isolated luminal progenitor (LP) cells from healthy reduction mammoplasties (see FIG. 1A) via FACS using the established markers CD49f and EpCAM⁴³ (see FIGS. 1B and 2A). The inventors ensured sample purity by re-analyzing sorted populations (see FIG. 2B) and only sorts with a purity of at least 99.5% were used for further experiments. As ECM surrogate, the inventors employed gels composed of collagen type I as outlined before^{35,44}.

In order to maintain luminal cell characteristics and to ensure robust rates of proliferation, the inventors used the breast cancer organoid medium (BCOM) as previously described⁴². BCOM was originally used for culturing mammary epithelial fragments in Matrigel and thereby supported the maintenance of the luminal as well as the basal cellular subset. The inventors observed that in combination with the collagen type I matrix, a fraction of single LP cells (CD49f^{high}/EpCAM^{high}) cultured in BCOM formed small cell clusters and gave rise to simple branched organoids with at least two branching points (see FIG. 2C/D). Accordingly, the inventors achieved a 12-fold increase in branched structure formation of single LP cells by the addition of 0.5% FCS to the medium (see Table 2), which was hereafter termed branched luminal organoid medium (BLOM). Other morphological shapes that occurred in both media were sticks and spheres (see FIG. 1C/D and FIG. 2C/D). Notably, when cells were cultured in Matrigel instead of collagen type I, no branched structure formation occurred (see FIG. 2E). Importantly, branched structure forming ability in collagen type I gels was not donor specific, but branched luminal organoids could be generated successfully from all donors tested (n=12, see FIG. 1E, Table 1). In line with previous observations, sorted single luminal mature cells (CD49f^{low}/EpCAM^{high}) barely proliferated when cultured in BLOM in collagen type I gels, whereas single basal cells (CD49f^{high}/EpCAM^{low}/CD10⁺) also generated branched structures (see FIG. 2F).

In summary, these data show that healthy primary LP cells in collagen type I matrix have the capacity to give rise to complex branched structures strongly resembling the branched morphology of low-grade NST (see FIG. 1F).

### Example 2: Branched Luminal Organoids Arise Clonally and Express Luminal Lineage and Polarization Markers

The inventors set out to further characterize LP-derived branched structures derived in BLOM focusing on frequency, morphogenetic steps and lineage marker expression. The inventors performed extreme limiting dilution analysis (ELDA) to first unravel the proportion of cells within the LP population that have structure forming potential⁴⁵. For this purpose, sorted LP cells were seeded in limiting dilution into collagen type I gels and their single-cell state was confirmed by light microscopy, thus indicating that arising structures were generated by one cell. Thereby, the inventors determined that overall between 1 out of 15 freshly sorted LP cells had the capacity to grow into a branched structure. This can be seen from the Table 6 given directly below.

TABLE 6
Extreme limiting dilution analysis (ELDA): determination of branched
structure-forming units (B-SFU)

| Number of cells | Positive gels/total gels | | |
|---|---|---|---|
| seeded | M36 | M44 | M50 |
| 5 | 1/6 | 3/6 | n/A |
| 10 | 2/6 | 4/6 | 2/6 |
| 20 | 5/6 | 5/6 | 4/6 |
| 40 | 6/6 | 6/6 | 5/6 |
| 80 | 6/6 | 6/6 | 6/6 |
| 160 | 6/6 | 6/6 | 6/6 |
| 320 | n/A | n/A | 6/6 |
| B-SFU | 1/15.1 | 1/9.52 | 1/20.7 |
| 95% CI | 1/8.85-1/26 | 1/5.53-1/16.7 | 1/11.8-1/36.5 |
| Mean B-SFU | | 1/15.11 | |

Data are mean with 95% confidence intervals (CI).

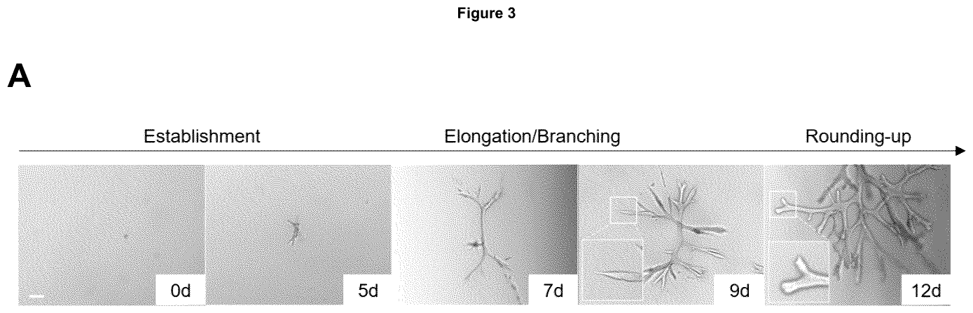

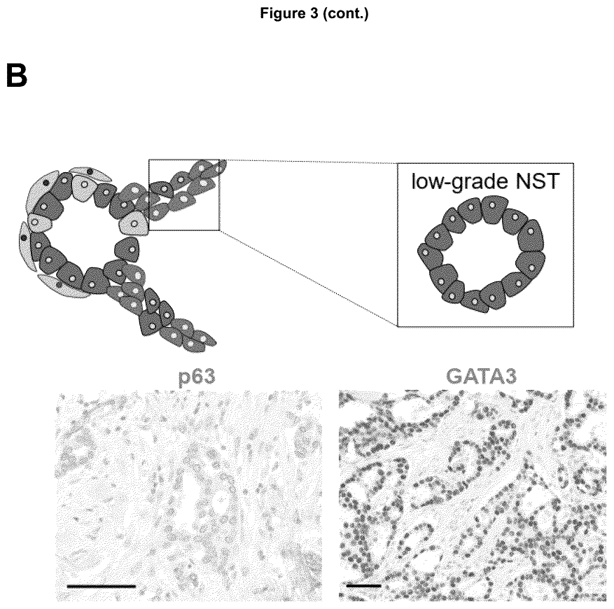

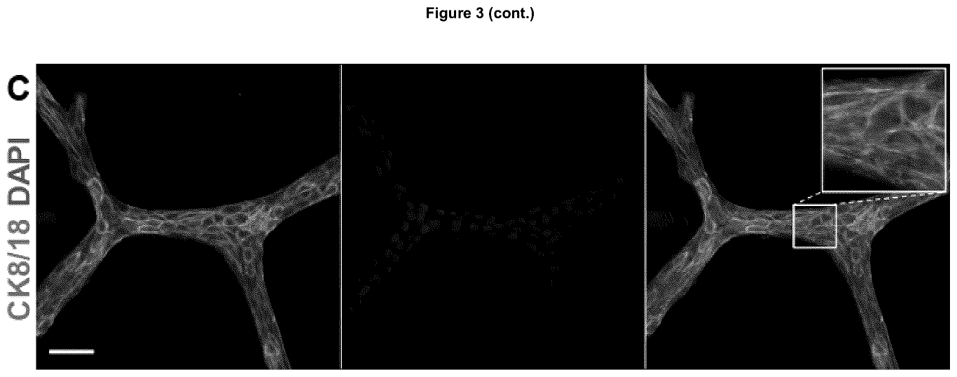

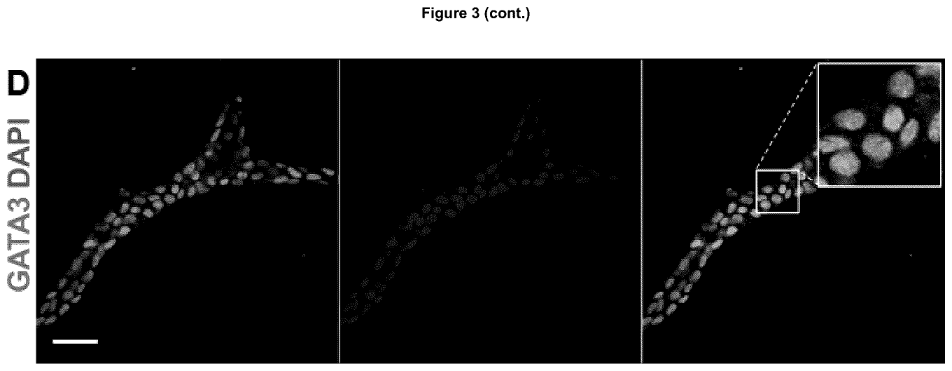

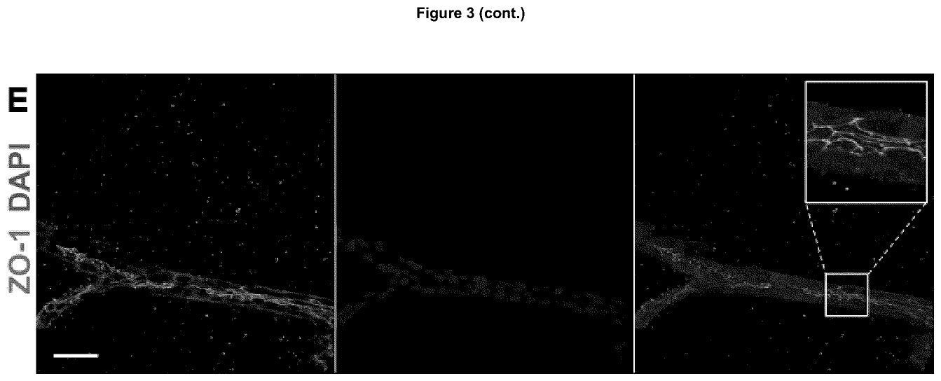

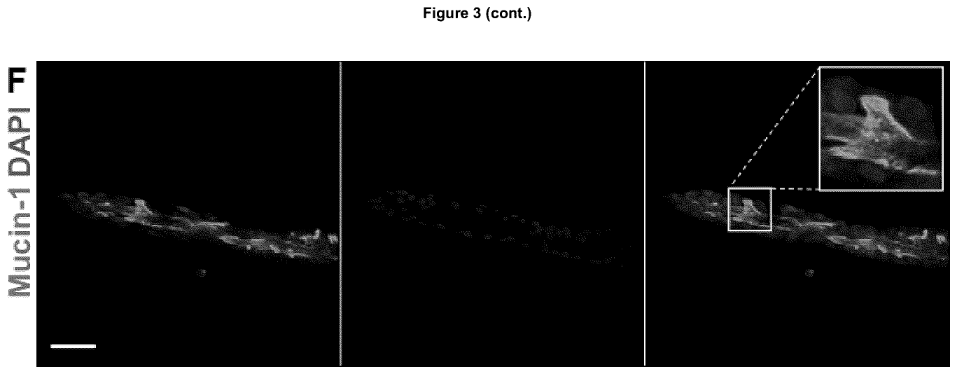

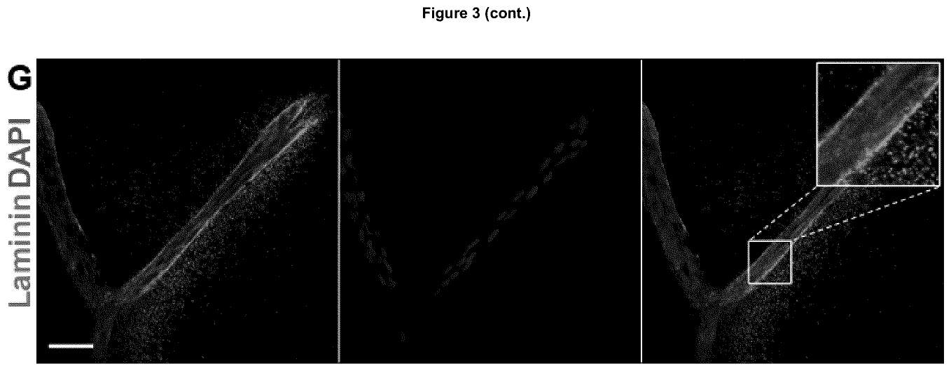

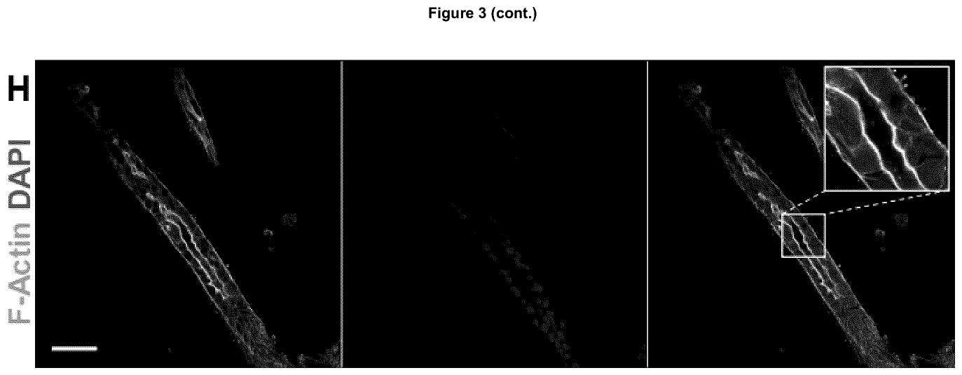

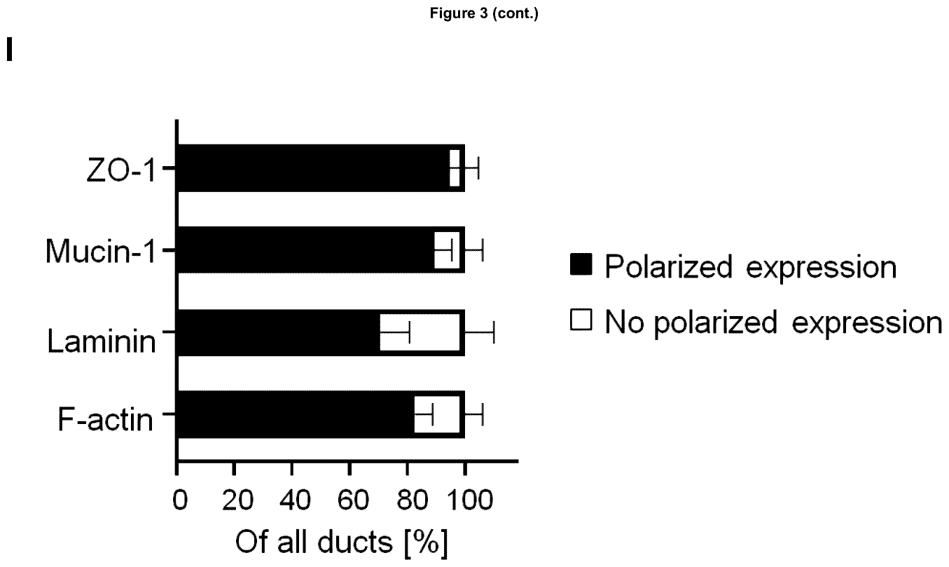

Through low seeding density and daily microscopic analysis the inventors could rule out that branched structures at a later stage resulted from merged organoids. Moreover, monitoring of organoid growth revealed different growth phases. During the establishment phase (day 1-5), single LP cells started proliferating and formed small cell clusters. Around day 5 of culture, small branches were formed, which further expanded into the matrix whereby the structure complexity continuously increased (elongation/branching phase, day 5-10). Finally, the inventors observed decreased branch elongation, accompanied by rounding up and thickening of the tips (days 11-13) (see FIG. 3A).

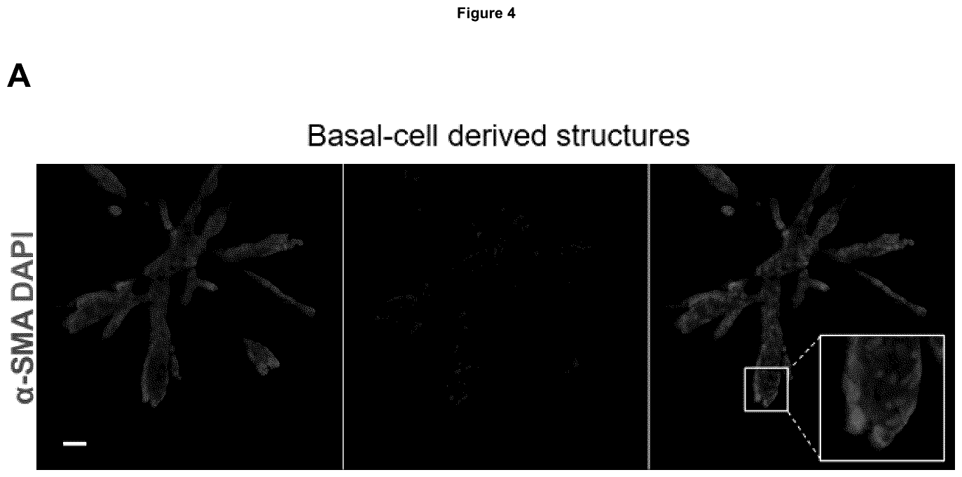

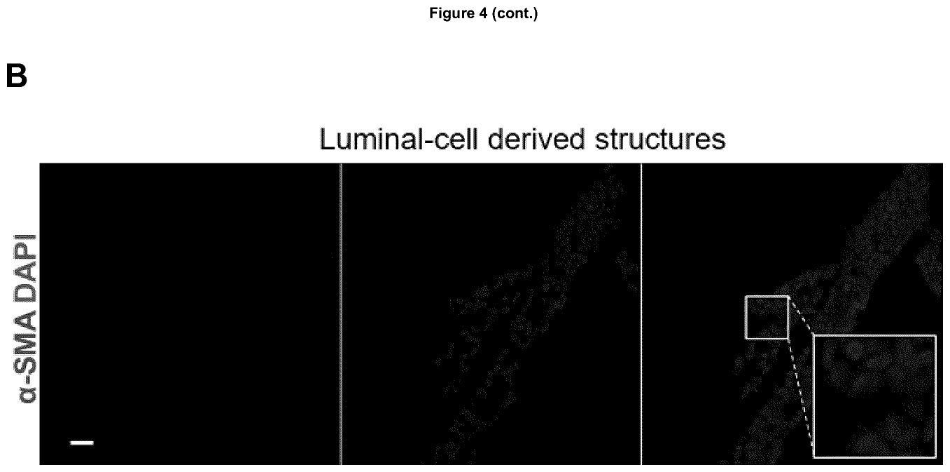

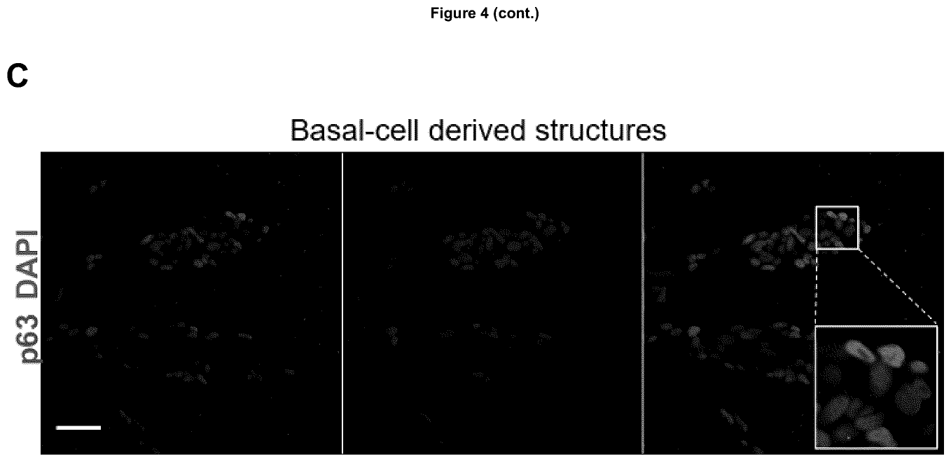

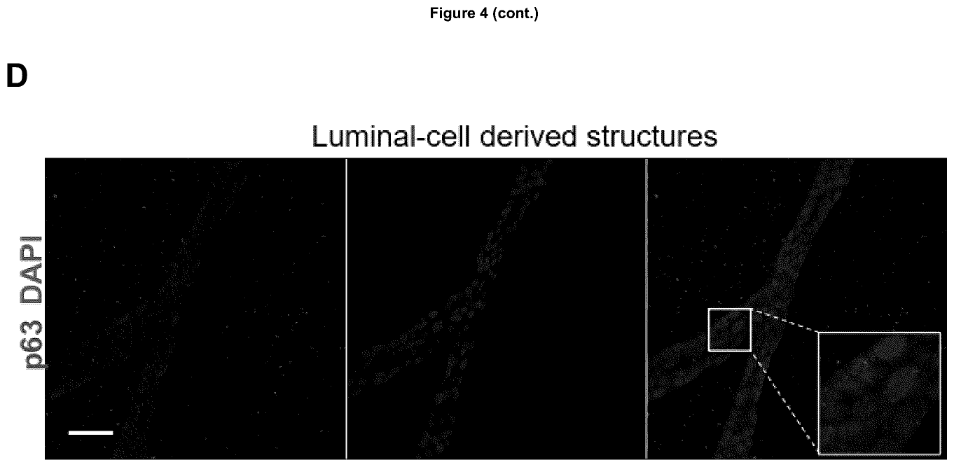

Building on the morphological resemblance to low-grade NST, the inventors next set out to assess lumen formation and luminal marker expression as hallmarks of low-grade NST pathology (see FIG. 3B). Those markers include CK8/18⁴⁶, GATA-3^{11,12}, ZO-1⁴⁷ and mucin-1⁴⁸. Confocal immunofluorescence of fully grown organoids (day 11-13) revealed expression of intermediate filaments CK8/18 within the cytoplasm (see FIG. 3C) and nuclear expression of transcription factor GATA-3 (see FIG. 3D) in all organoids examined. Throughout the organoids, the inventors found a non-continuous lumen mostly framed by a single cell layer (see FIG. 3C-H). Previously, correct apical-basal polarization of the epithelium has been shown to be a prerequisite for lumen formation during collective invasion of epithelial cells⁴⁹. In line with this notion, the inventors found tight junction protein ZO-1 at an apical position indicating correct apical-basal polarization (see FIG. 3E/I). Furthermore, the inventors discovered mucin-1, a marker of luminal differentiation, at the apical side of most organoids (see FIG. 3F/I). To corroborate this notion, the inventors examined the non-lineage specific polarization markers laminin and F-actin. The inventors observed that the basolateral side of LP-derived organoids was partially covered by the basal lamina component laminin (see FIG. 3G/I), while an accumulation of F-actin towards the apical side was found at locations where a lumen was formed (see FIG. 3H/I). In addition, the inventors investigated the expression of basal lineage markers p63 and alpha smooth muscle actin (α-SMA)³⁹, which are typically absent in NSTs (see FIG. 3B). The inventors used basal-cell-derived structures as a positive control, which reliably expressed p63 and α-SMA (see FIG. 4A/C). In contrast, neither p63 nor α-SMA was detected within luminal organoids (see FIG. 4B/D).

In summary, these results show for the first time that upon transplantation into a collagen type I environment, a large fraction of healthy primary LP cells bears the potential of growing out into clonal, complex branched structures. Doing so, luminal cells maintained their lineage marker expression and mature ducts displayed apical-basal polarity as well as lumen formation. As a result, final organoids resemble low-grade NST.

### Example 3: LP-Derived Branched Organoid Formation Requires Inhibition of ROCK-Myosin II Signaling

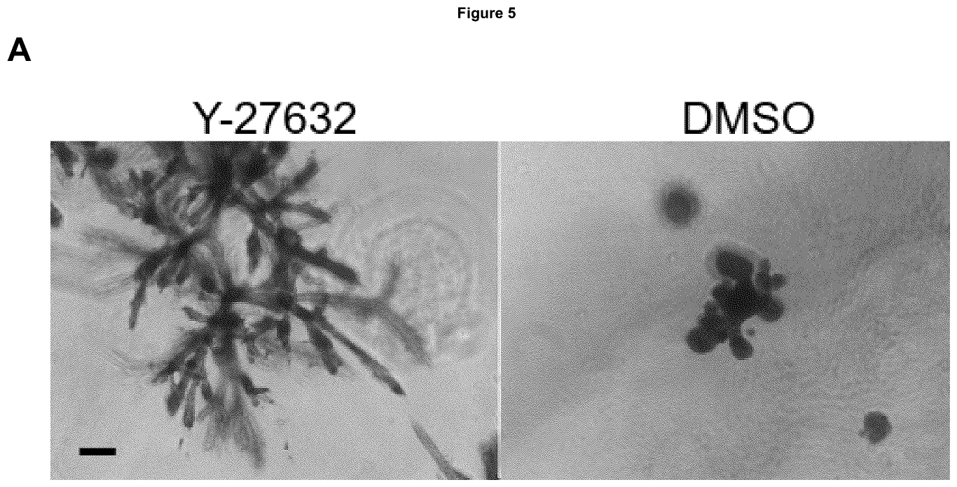

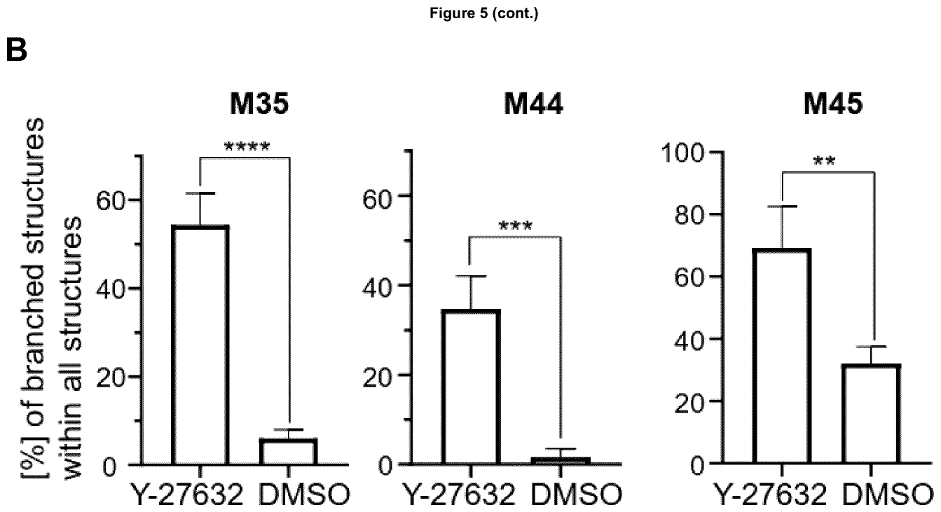

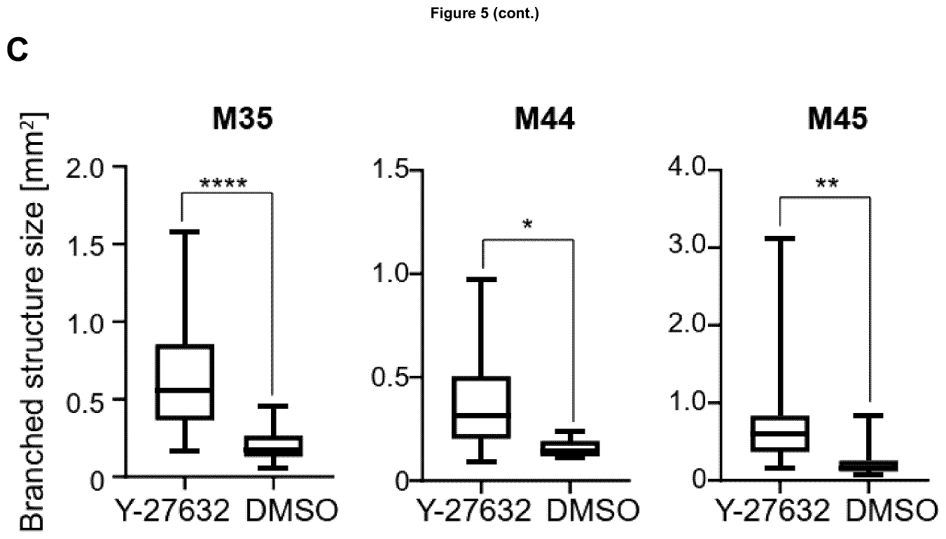

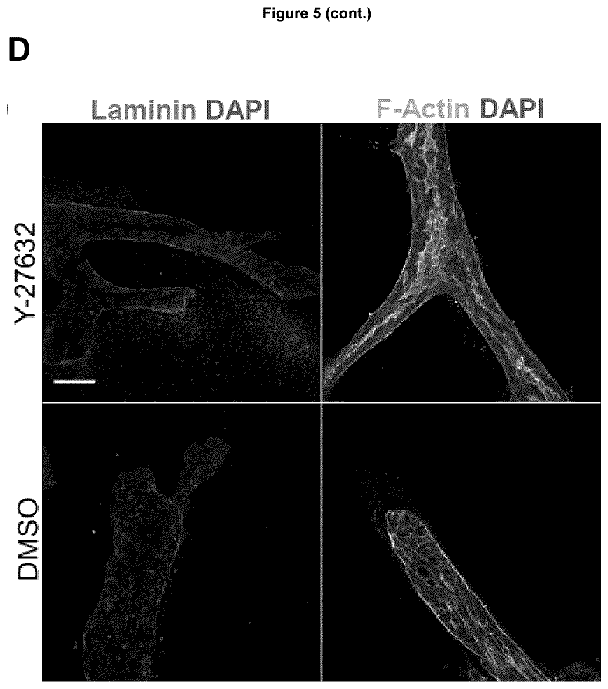

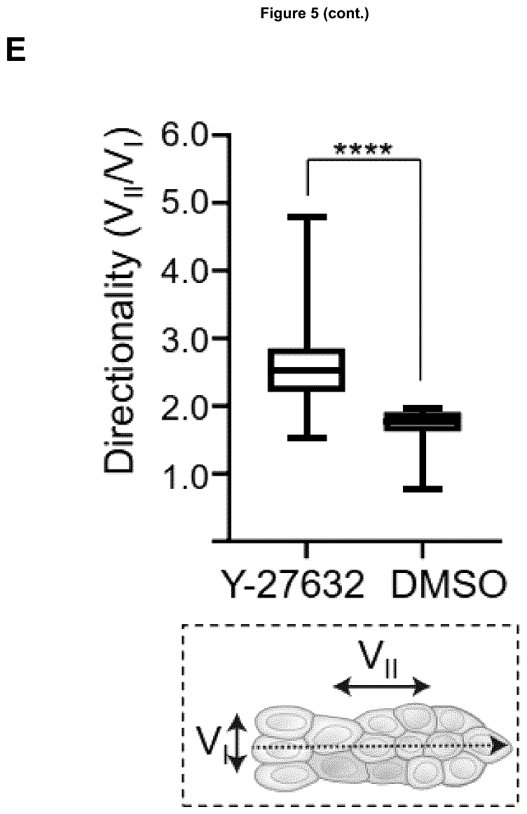

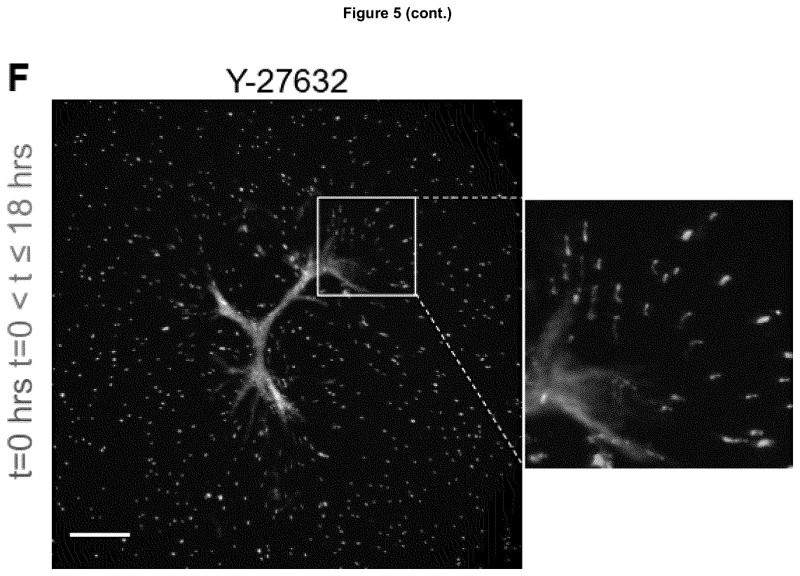

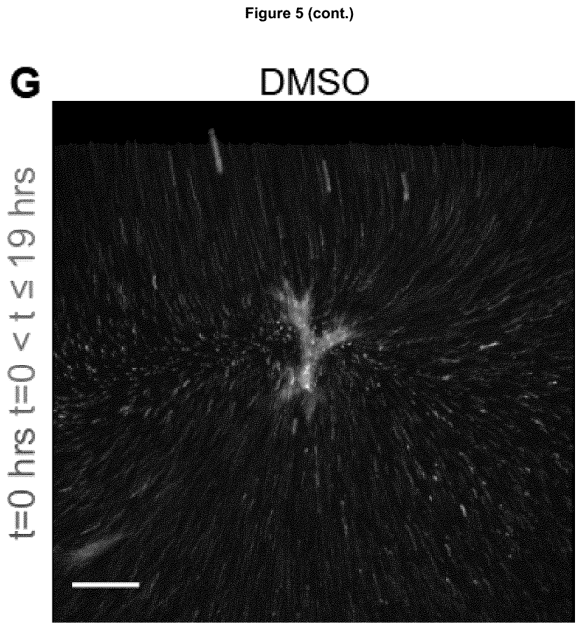

In contrast to previous experimental setups, where LP cells cultured in collagen type I gels gave rise to small spheres^{35,50}, the inventors observed the formation of highly polarized, branched structures. Importantly, it has been shown that invasion as well as apical-basal polarization of epithelial structures in collagen type I matrices can require regulation of RhoA-ROCK-myosin II signaling via inhibition of the Rho-associated protein kinase (ROCK)^{51,52}. Thus, the inventors concluded, that the ROCK inhibitor Y-27632 might play a critical role for branched structure formation and polarisation of LP-derived organoids. Indeed, depriving the medium of Y-27632 prevented the formation of elongated branched structures almost completely (see FIG. 5A). In detail, the removal of Y-27632 led to a decrease in branched structure formation by 79%. Instead, a majority of cells grew out as spheres and sticks (see FIG. 5B and FIG. 6A). Furthermore, the rarely emerging branched structures were significantly smaller in size (−66%) (see FIG. 5C) and showed a reduced branching complexity. Specifically, only 6% of organoids showed tertiary or even more complex branches, while 65% of control organoids were this complex (see FIG. 6B). In addition, immunofluorescence revealed that polarization was disturbed in the absence of ROCK inhibition (see FIG. 5D). Specifically, LP structures derived in the absence of Y-27632 lacked lumen formation, and neither laminin deposition at the basolateral side nor F-actin accumulation at the apical side was observed. Finally, the inventors set out to provide further support for inhibition of the ROCK-myosin II pathway as a prerequisite for branched structure formation by targeting a different segment of this pathway. To do so, the inventors replaced Y-27632 by para-amino blebbistatin (PAB), an inhibitor of the ROCK downstream target myosin II. Indeed, PAB was able to induce a phenotype similar to the one created by Y-27632 (see FIG. 6C/D).

Next, the inventors wished to assess the dynamics of the process required for the formation of LP-derived branched structures and its dependency on ROCK inhibition. For this purpose, the inventors performed live-cell confocal microscopy during the organoid elongation phase, focusing on the cellular dynamics within branched organoids and their interaction with the surrounding collagen matrix. Specifically, the inventors monitored cellular movements within LP-derived branched organoids in the presence and absence of Y-27632 using nuclear labelling. Thereby, the inventors observed that under ROCK inhibition, cell migration was primarily directed outwards in parallel to the axis of the extending branch. This directionality was significantly diminished in conditions without ROCK inhibitor. Here, the parallel (V_{II}) and orthogonal (V_{I}) velocity, which is directed perpendicular to the branch axis, were more similar, resulting in a less directed movement (see FIG. 5E). Furthermore, to visualize cellular interaction of the cells with the surrounding ECM, the inventors conducted live cell imaging of organoids in collagen type I gels, spiked with fluorescent tracer particles/beads. Doing so, the inventors observed that in the presence of a ROCK-inhibitor, bead displacement and therefore contractility was not completely abolished, but limited to the front of elongating branches (see FIG. 5F). By contrast, in the absence of ROCK-inhibition, the inventors observed strong, generalized bead displacement towards the organoids suggesting high cellular contractility (see FIG. 5G).

Together, these results show that in normal LP cells, reduction of the ROCK-myosin II signaling can enable directed contractility and migration, resulting in branching morphogenesis and concomitant apical-basal polarization in collagen type I gels.

### Example 4: Ductal Organoids are Generated Through Invasive Branching Morphogenesis

The inventors hypothesized that in order to undergo branching morphogenesis, resulting in the observed low-grade carcinoma morphology, LP cells need to actively and collectively invade the ECM. Active invasion requires remodelling of the mammary ECM, which has often been described to strongly rely on allied stromal cells⁵³. Importantly, the engagement with the ECM during luminal organoid formation as described above was suggestive of matrix remodelling capacity within healthy LP cells. Therefore, in order to understand invasive mechanisms exploited by LP cells, the inventors first focused on matrix topography.

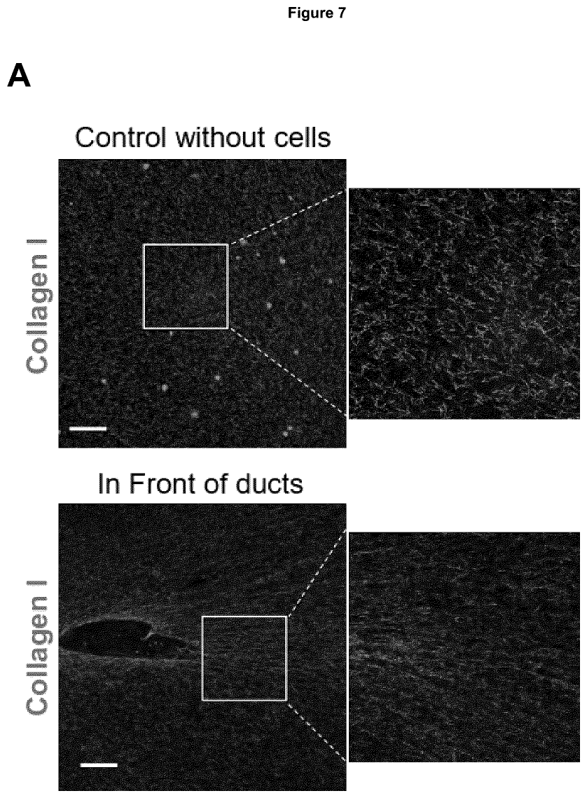

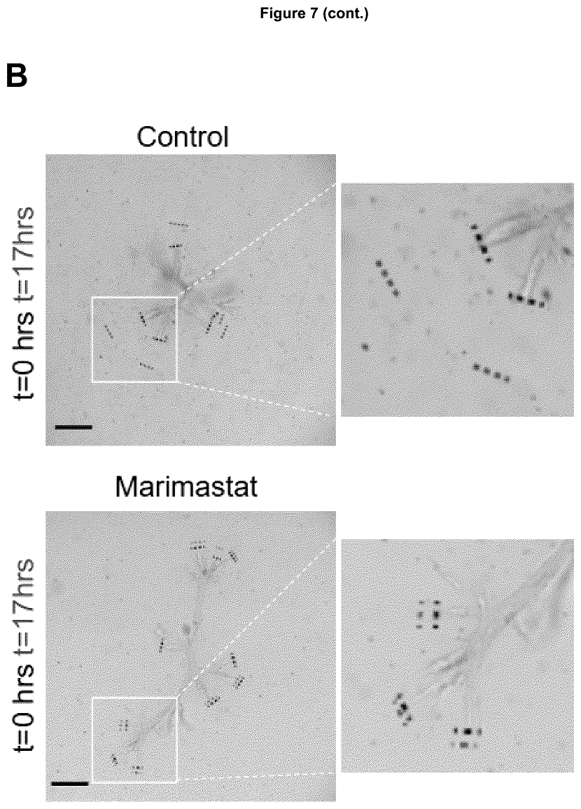

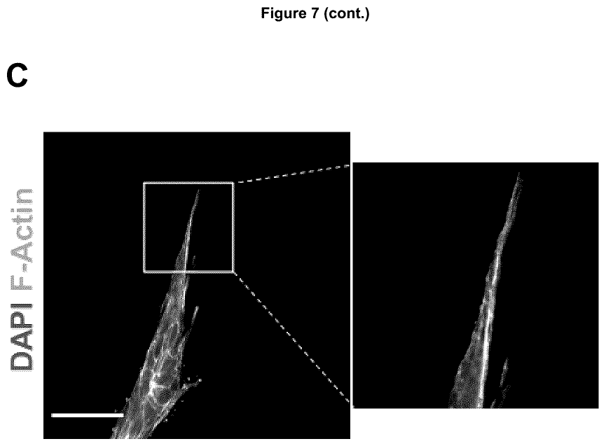

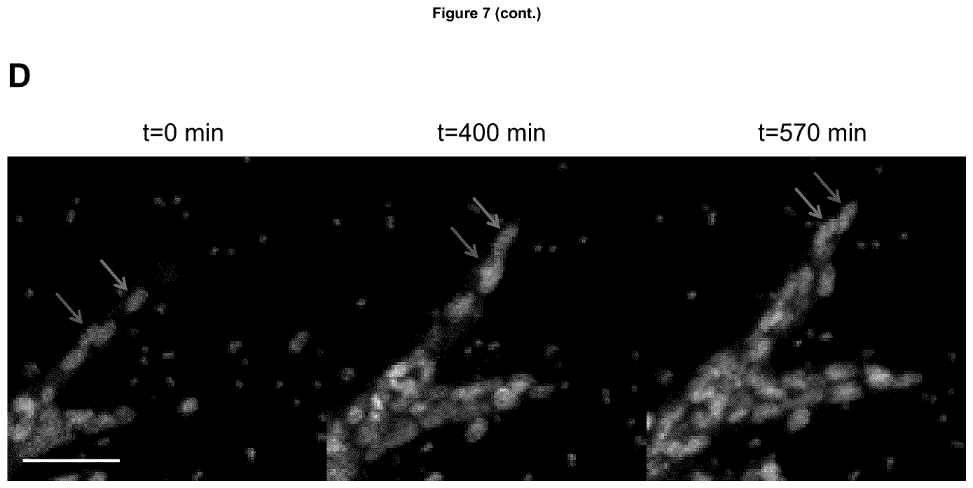

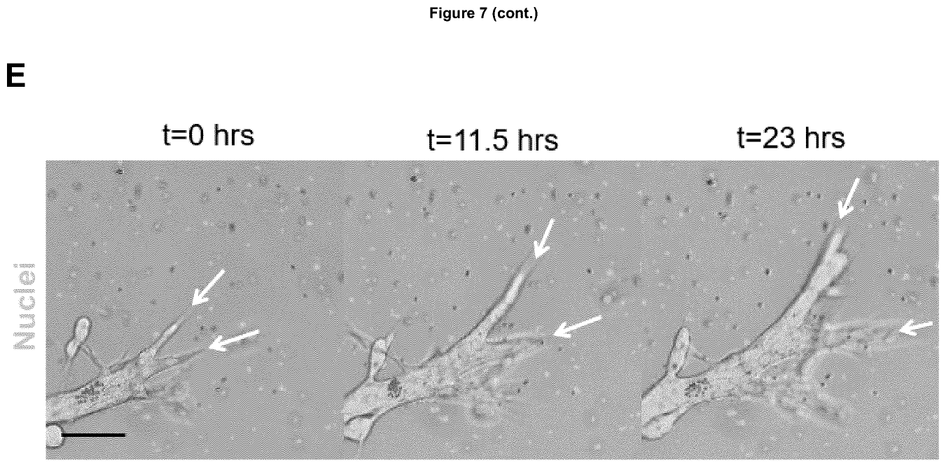

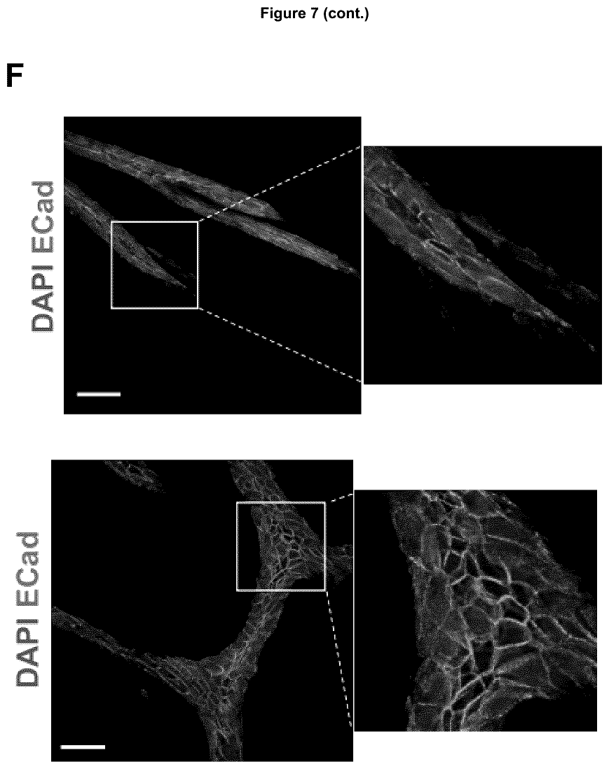

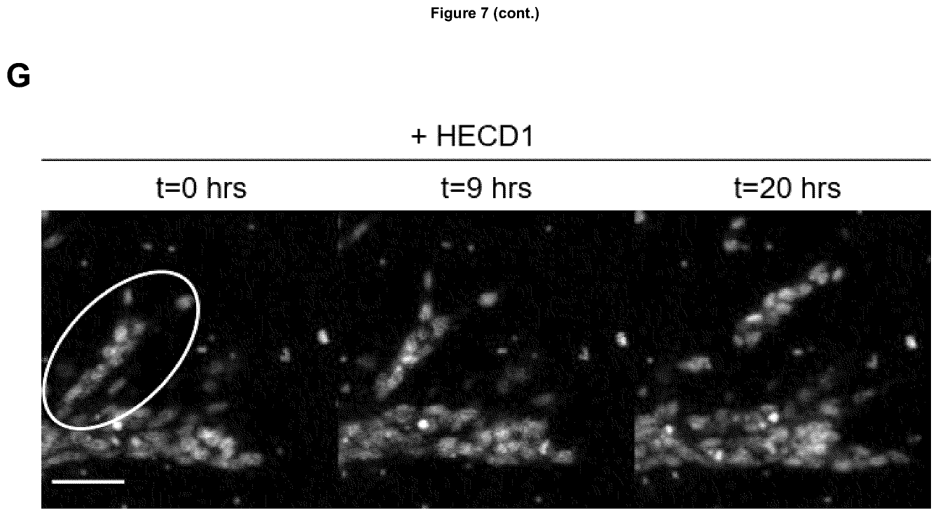

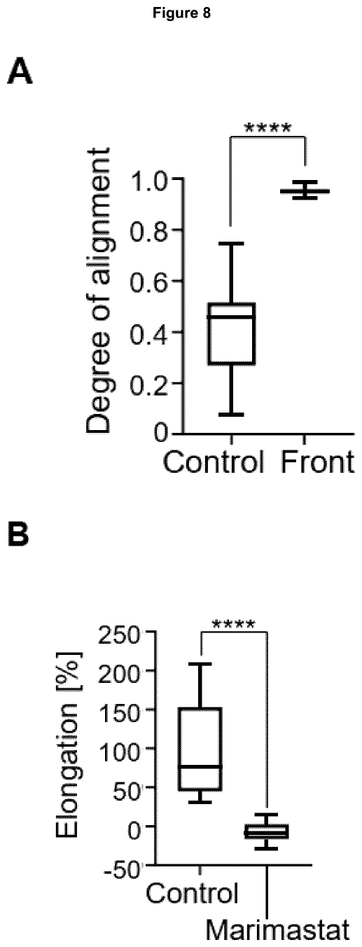

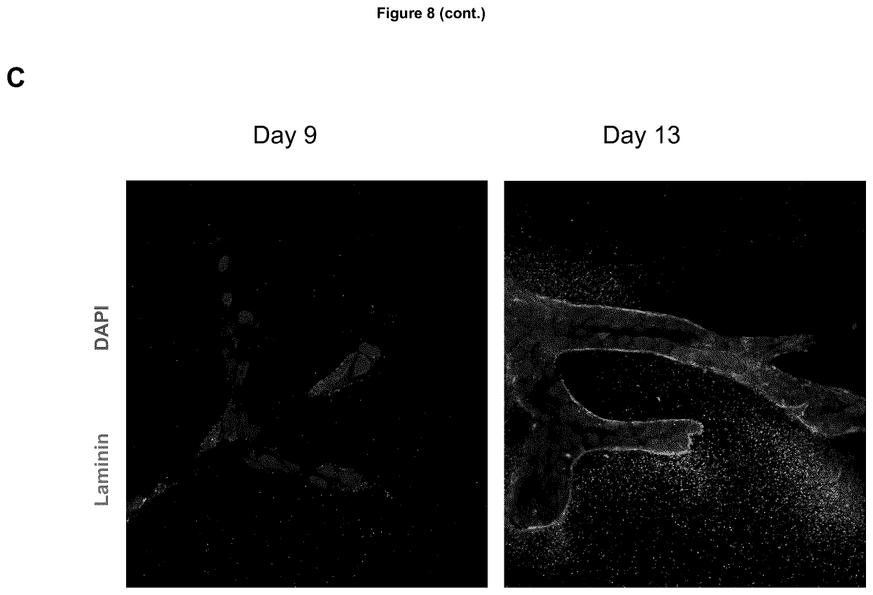

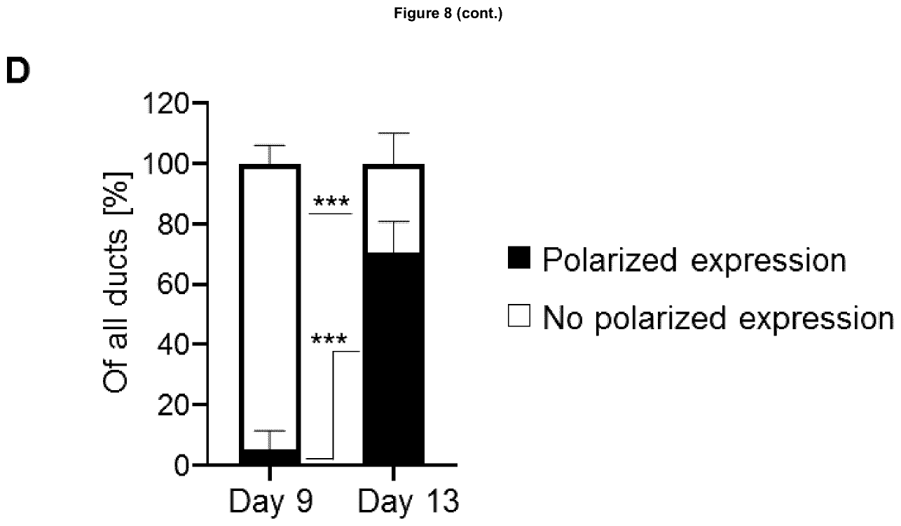

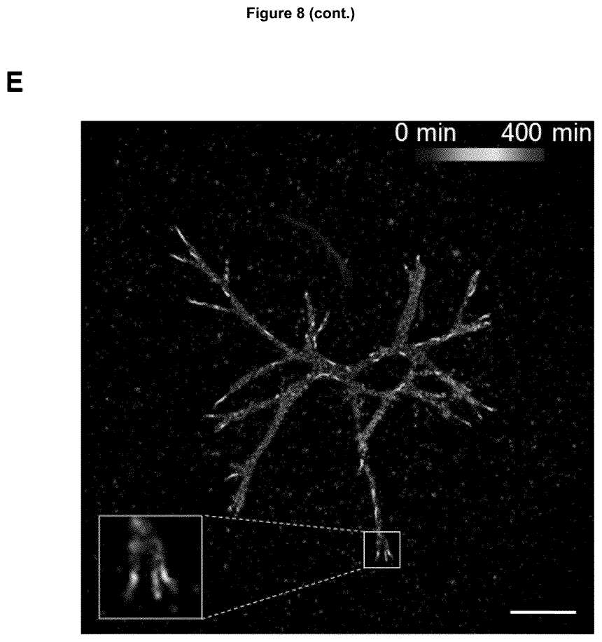

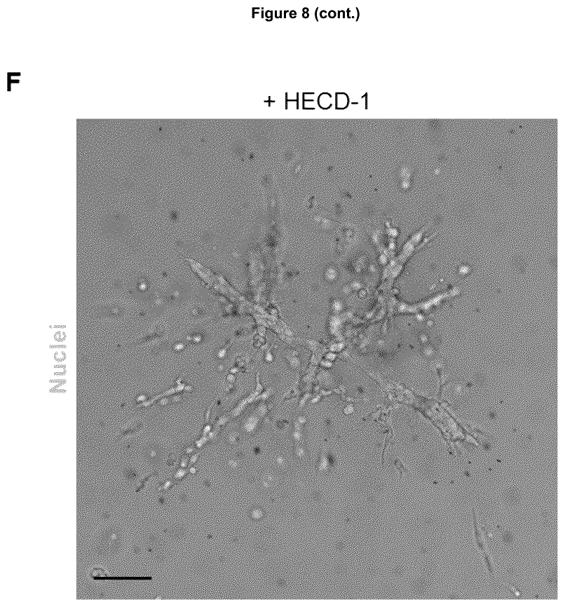

Normal mammary glands are typically surrounded by curly, anisotropic collagen fibers⁵⁴. Upon tumor initiation, collagen fibers are linearized, which promotes migration and invasion of breast cancer cells along aligned fibers. This alignment has been shown to be facilitated by fibroblasts or the invading carcinoma cells themselves^{30,32,54}. Similarly, using fluorescently labelled collagen type I, the inventors observed an alignment of collagen fibers in front of the extending organoid branches. By contrast, collagen fibers in the periphery of the organoid showed no preferred orientation (see FIG. 7A and FIG. 8A). Importantly, it is well-known that fiber alignment requires ROCK-mediated cellular contractility^{30,65}. This further supports the notion that ROCK-inhibition as employed in the model of the inventors does not completely abolish cellular contractility, but rather reduces it to a level enabling directed movement of cells as well as rearrangement of collagen fibers. Another crucial aspect of matrix remodelling during invasion is active degradation by matrix-metalloproteinases (MMPs), which has also been observed as a competence of stromal as well as epithelial cells^{56,57}. Accordingly, live cell imaging revealed that treatment with the MMP inhibitor Marimastat during the branch elongation phase, stopped the branch tips from further invading the matrix. At the same time, cell movements and divisions persisted within the organoid body, thereby excluding a general cytotoxic effect of Marimastat treatment (see FIG. 7B and FIG. 8B). Of note, the inventors found that during the observed elongation phase, the distinct expression of laminin as described for fully grown organoids was not established yet. This finding helps to explain how direct interaction of the invading cells with the collagenous ECM is enabled (FIG. 8C/D).

As the inventors observed matrix remodelling mainly at the tips of elongating branches, the inventors assumed that collectively invading ducts are led by leader cells. The inventors found that during elongation phase, there was typically a single cell at the tip of each duct, which exhibited an extended morphology characterized by filopodia-like membrane protrusions (see FIG. 7C). Matching this observation, live cell imaging revealed that during elongation phase there was one cell at the tip of each duct, which continuously grew outwards and remained at the front position over several hours (see FIG. 8E). However, the inventors also observed that the leader cell was not per se specialized, but could be exchanged by a cell from the structure body (see FIG. 7D). The observed elongation phase was characterized by continuously increasing complexity of the structure. The inventors found that new branches arose mainly via bifurcation (21 out of 26 events, 4 donors) when two neighbouring leader cells with differently oriented filopodia-like protrusions were present (see FIG. 7E). Rarely, the inventors also observed the formation of new branches by cells growing out of the structure body (5 out of 26 events, 4 donors).

Transfer of leader cell generated forces along epithelial cohorts during collective invasion has been described to depend on cadherin-mediated cell junctions⁵⁸. Immunofluorescence confirmed that cells of invading structures were tightly connected with the leader cells and with each other via E-cadherin (see FIG. 7F). The importance of E-cadherin mediated cell-junctions during this process was determined by treating structures with a function blocking antibody (HECD-1). Live cell imaging of HECD-1 treated organoids during elongation phase revealed dissolution of invasive branching morphogenesis with single cells or even whole branches losing contact to the structure body (see FIG. 7G). Consequently, the inventors saw scattering of the structures (see FIG. 8F).

Together these results show that normal LP cells actively invade into the ECM via matrix remodeling. Thereby, interchangeable leader cells guide invasive branching morphogenesis. Finally, tight cell-cell connections with the leader cell, mediated, at least in part by E-cadherin, ensure duct integrity during collective invasion.

### Example 5: Deletion of E-Cadherin Results in ILC-Like Morphology

The strong impact on organoid morphology observed upon E-cadherin inhibition is in line with the critical role of E-cadherin status in invasive cancer formation. In vivo, E-cadherin status is the main discriminator between NST and invasive lobular carcinoma (ILC), a specific, morphologically distinct subtype of invasive cancer. While NSTs maintain E-cadherin, in ILC, the full loss of genetic function and therefore protein expression is typically observed^{14,15}.

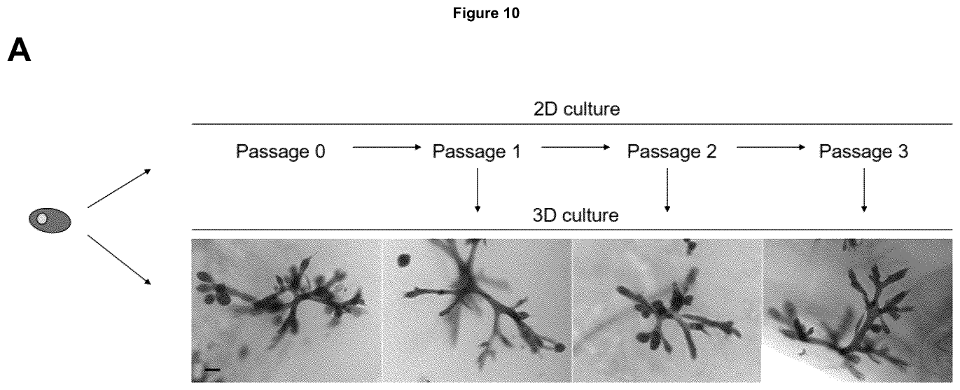

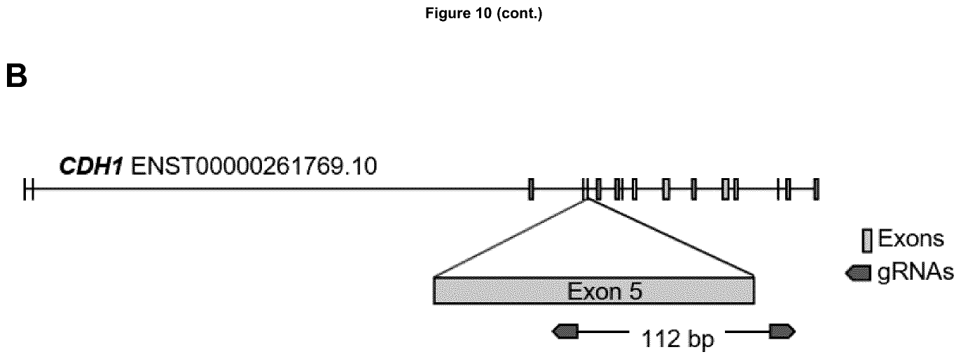

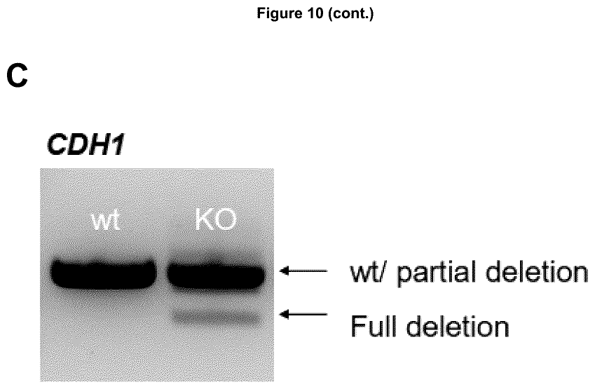

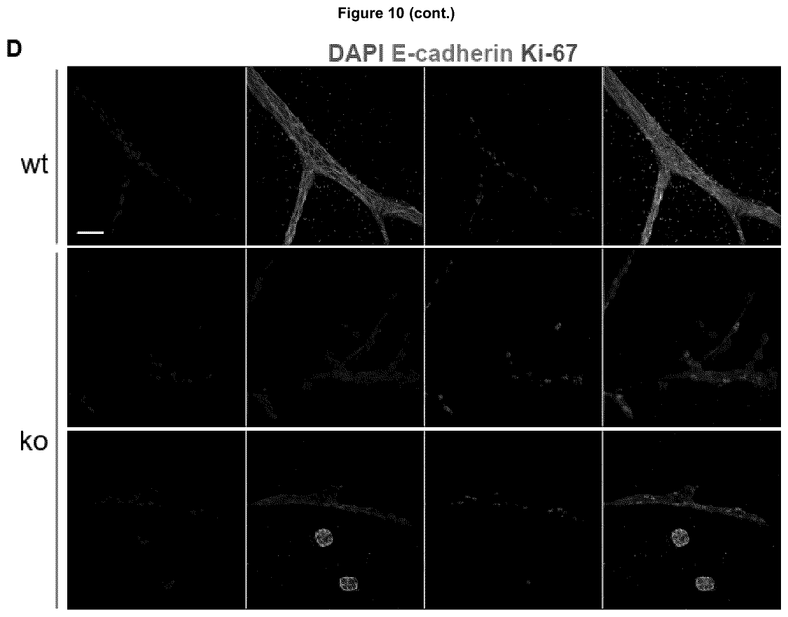

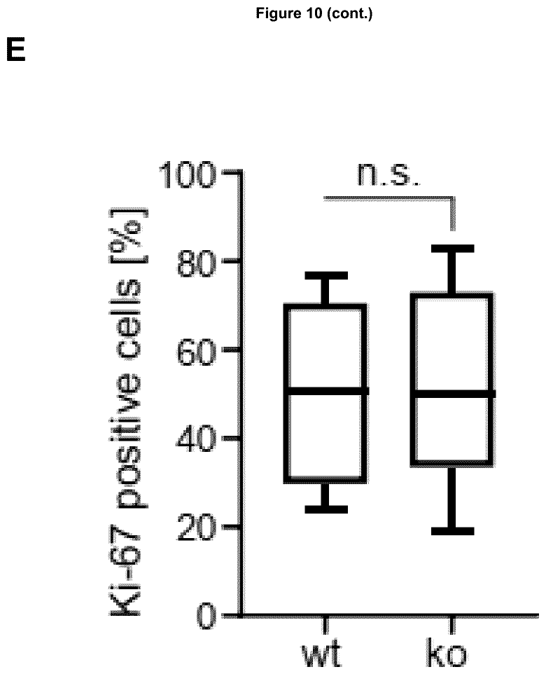

Based on these considerations, the inventors set out to recapitulate the full deletion of E-cadherin encoded by CDH1 with the CRISPR-Cas9 system. Thereby, the inventors took advantage of the clonality of outgrowing organoids in order to create clonal knockout (KO) structures. To efficiently perform the KO, the inventors tested whether organoid forming potential of LP cells was maintained during prior 2D culture. The inventors determined that 3D branched organoids could be generated even after three passages in 2D culture (see FIG. 10A).

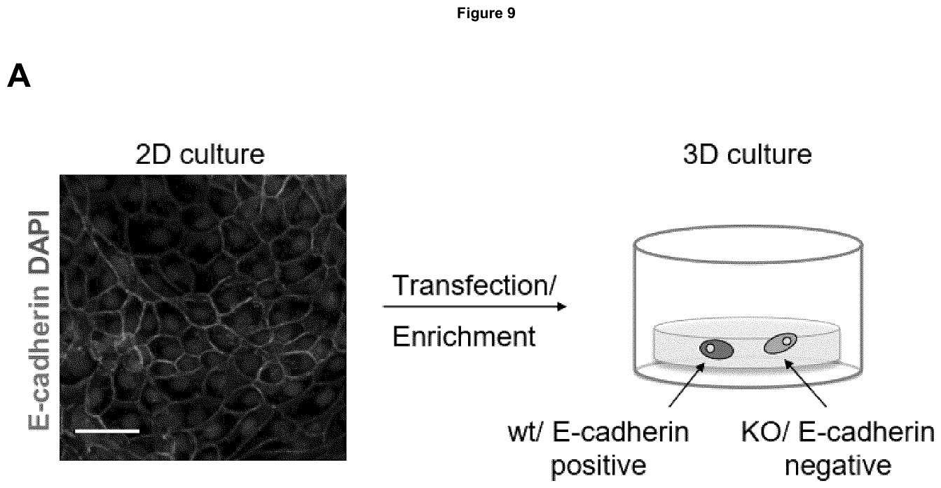

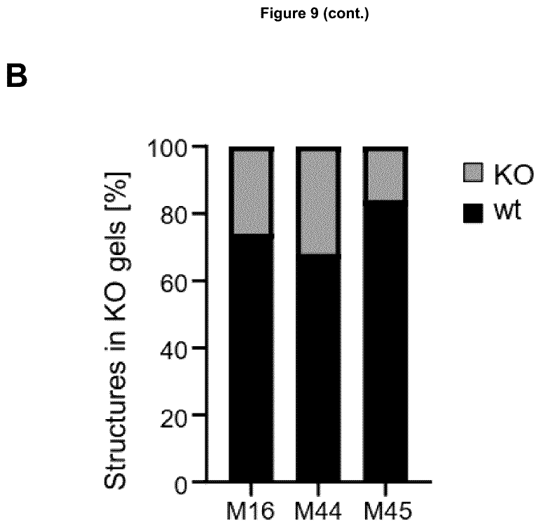

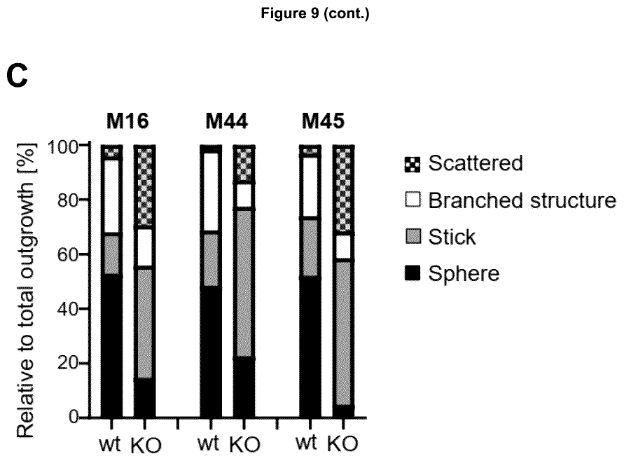

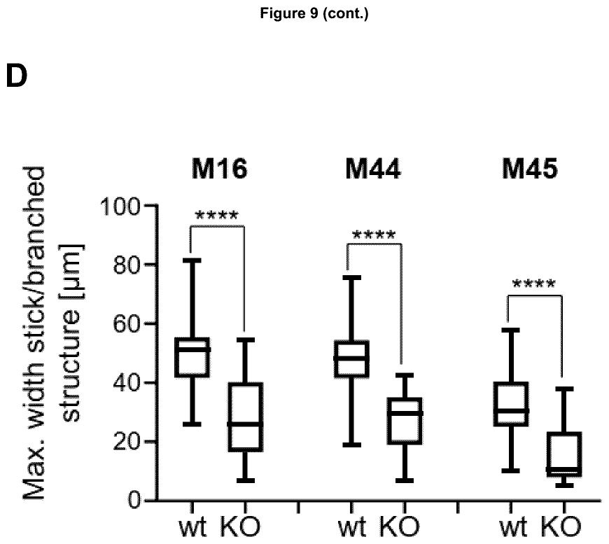

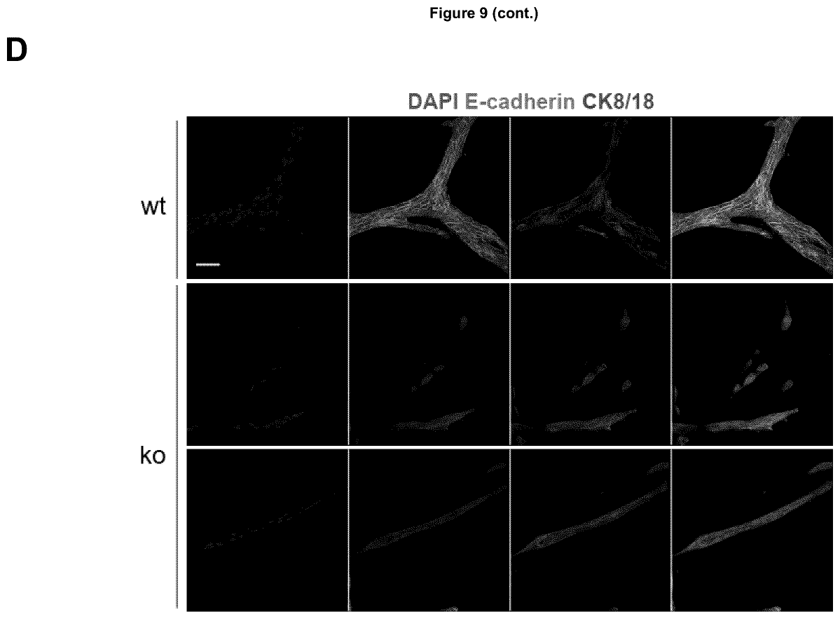

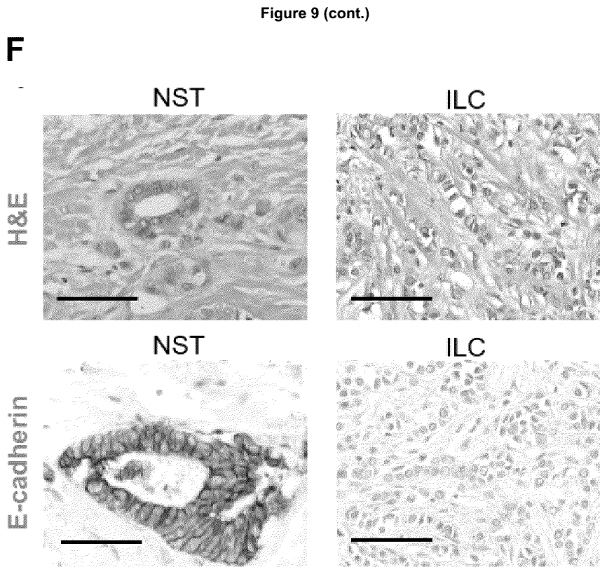

After transfection of LP cells in 2D and enrichment for successfully transfected cells via a GFP tagged Cas9 plasmid using FACS, single LP cells were seeded into collagen type I gels (see FIG. 9A). Upon KO with two gRNAs (SEQ ID NOs: 1 and 2) framing a section of exon 5, the inventors saw a partial deletion on the genetic level within the sorted population (see FIG. 10B/C). Due to the clonal growth of single cells within the assay, the inventors could differentiate between wild-type (wt) and KO structures by E-cadherin immunofluorescence. Overall, the inventors observed a mixture of wt and KO structures with around 25% of structures bearing the KO (see FIG. 9B). Notably, in control gels with empty-vector transfected cells, no E-cadherin negative structures were observed (data not shown). Importantly, the phenotypes of fully grown wt and KO structures were strikingly different. Wt cells grew into sticks, spheres and complex branched structures of the previously described morphology. In contrast, KO structures were typically thinner and often showed stick-like instead of branched morphology. Several KO structures showed partial loss of cell-cell contact and were scattered throughout the gel. In addition, a decrease in sphere formation was observed (see FIGS. 9C, 9D and 9E). In order to exclude that the aberrant morphology was the result of loss of proliferative capacity, the inventors performed a Ki67 staining and determined that KO and wt structures showed similar rates of proliferation (see FIG. 10D/E).

Taken together, the morphology the inventors observed upon KO of E-cadherin was reminiscent of the growth pattern that ILCs exhibit in vivo, which is characterized by absence of duct formation and cells penetrating the matrix as single cells or thin files of cells (see FIG. 9F)⁵⁹. Thus, the inventors concluded that knock-out of a gene relevant for invasive branching morphogenesis has characteristic impact on the resulting phenotype of the in vitro method/model of the present invention.

### SUMMARY AND DISCUSSION OF THE EXAMPLES

When cultured in collagen type I gels under defined conditions, up to 10% of freshly isolated normal LP cells generated complex multicellular organoids through invasive branching morphogenesis. Considering that tissue isolation and FACS prior to 3D culture can drastically reduce the viability of primary LP cells, the inventors hypothesized that matrix invasion potential does not represent the ability of a rare luminal subset. Rather, the results of the inventors indicate that almost every single LP cell bears matrix invasion potential, which can be triggered under suitable conditions that allow for sufficient proliferative capacity. This supports the theory that invasion potential is already existent in pre-invasive malignancies rather than being unlocked in the luminal compartment as a consequence of specific genetic aberrations¹⁸⁻²⁰.

Morphologically, the organoids arising from unmodified LPs resemble low-grade NST. Even though invasive behaviour per se has not generally been connected to certain genetic aberrations in the luminal population, specific perturbations impact invasive branching morphogenesis and are strongly associated with breast cancer subtype. Our single-cell based assay allows insertion and precise quantification of phenotypes connected to genetic aberrations as evident by the analysis of an E-cadherin KO phenotype. Here, the KO morphology mirrors the loss of duct formation and thereby closely resembles clinical samples of the ILC subtype. Therefore, the system is a useful tool for unravelling gene specific impact on invasive subtype and screening purposes based on phenotype.

In the 3D organoid model of the inventors, matrix invasion of LP cells was highly dependent on ROCK inhibition, suggesting that an activation of the ROCK-myosin II signaling cascade represents an invasion-suppressing response of LP cells upon contact to the collagenous ECM. A similar observation has been reported recently for murine cells in vivo where it was shown that the mere contact to the ECM did not result in luminal cell invasion. However, invasion was enabled once the functionality of actomyosin contractility regulator MYPT1 was reduced within the luminal cells⁶⁰. This phenomenon has further been reported in in vitro studies with immortalized normal-like mammary epithelial cells³⁴ as well as epithelial kidney cells⁵¹. In both cases, ROCK-inhibition enabled cellular invasion into a collagen type I matrix. In the model of the present invention, the inventors observed residual contractility even upon ROCK-inhibition as indicated by spatially restricted bead displacement and collagen fiber alignment, which in turn further supports directional migration^{54,55}. In line with these considerations, Schipper et al. showed that a full obstruction of MYPT1 function resulted in a loss of invasive capacity in vivo. Together, these results highlight the importance of a tightly balanced actomyosin contractility for luminal cell invasion rather than a full obstruction⁶⁰.

The ECM invasion process is based on complex interactions between epithelial and stromal cells and the surrounding matrix. Stromal cells, particularly, cancer-associated fibroblasts have been described as matrix remodelling drivers of invasion^{53,61,62}. The work of the inventors put the spotlight exclusively on the interaction of luminal cells with the surrounding collagen matrix, which in vivo only occurs once the basal cell layer and basement membrane barrier are disrupted. Nevertheless, the present invention shows that once the contact between LP cells and collagen type I is established, invasion relevant processes were executed by luminal cells themselves without the requirement for genetic aberrations or for stromal support.

According to the present invention, reduced contractility may be required for invasive branching morphogenesis of luminal cells. Nevertheless, understanding of how the same mechanism could be utilized in vivo during invasion of malignant luminal cells is lacking. The inability to identify universal aberrations between invasive and in situ carcinomas might hint towards participation of stromal factors for the reduction of actomyosin contractility. Future studies in which the collagen gels are complemented with cancer associated stromal cells will help to elucidate the relevance of stromal interactions during luminal cell invasion. Thus, by furthering the knowledge on luminal cell invasion, the present invention helps to find new means for early diagnosis and prevention of invasive cancers arising from the LP subset.

### REFERENCES

1. Molyneux, G. et al. BRCA1 Basal-like Breast Cancers Originate from Luminal Epithelial Progenitors and Not from Basal Stem Cells. Cell Stem Cell 7, 403-417 (2010).
2. Tao, L., van Bragt, M. P. A. & Li, Z. A Long-Lived Luminal Subpopulation Enriched with Alveolar Progenitors Serves as Cellular Origin of Heterogeneous Mammary Tumors. Stem Cell Reports 5, 60-74 (2015).
3. Proia, T. A. et al. Genetic Predisposition Directs Breast Cancer Phenotype by Dictating Progenitor Cell Fate. Cell Stem Cell 8, 149-163 (2011).
4. Lim, E. et al. Aberrant luminal progenitors as the candidate target population for basal tumor development in BRCA1 mutation carriers. Nat Med 15, 907-913 (2009).
5. Lopez-Garcia, M. A., Geyer, F. C., Lacroix-Triki, M., Marchió, C. & Reis-Filho, J. S. Breast cancer precursors revisited: molecular features and progression pathways. Histopathology 57, 171-192 (2010).
6. Li, C. I., Anderson, B. O., Daling, J. R. & Moe, R. E. Trends in incidence rates of invasive lobular and ductal breast carcinoma. JAMA 289, 1421-1424 (2003).
7. Bloom, H. J. G. & Richardson, W. W. Histological Grading and Prognosis in Breast Cancer. Br. J. Cancer 11, 359-377 (1957).
8. Contesso, G. et al. The importance of histologic grade in long-term prognosis of breast cancer: a study of 1,010 patients, uniformly treated at the Institut Gustave-Roussy. J. Clin. Oncol. 5, 1378-1386 (1987).
9. Elston, C. W. & Ellis, I. O. Pathological prognostic factors in breast cancer. I. The value of histological grade in breast cancer: experience from a large study with long-term follow-up. Histopathology 19, 403-410 (1991).
10. Patey, D. H. & Scarff, R. W. Histology in the prognosis of carcinoma of the breast. Lancet 1, pp-801-804 (1928).
11. Guo, Y. et al. Prognostic and clinicopathological value of GATA binding protein 3 in breast cancer: A systematic review and meta-analysis. PLOS One 12, e0174843 (2017).
12. Naylor, M. J. & Ormandy, C. J. Gata-3 and mammary cell fate. Breast Cancer Res. 9, 302 (2007).
13. Taylor-Papadimitriou, J. et al. Keratin expression in human mammary epithelial cells cultured from normal and malignant tissue: relation to in vivo phenotypes and influence of medium. J Cell Sci 94 (Pt 3), 403-13 (1989).
14. Gamallo, C. et al. Correlation of E-cadherin expression with differentiation grade and histological type in breast carcinoma. Am J Pathol 142, 987-993 (1993).
15. Moll, R., Mitze, M., Frixen, U. H. & Birchmeier, W. Differential loss of E-cadherin expression in infiltrating ductal and lobular breast carcinomas. Am J Pathol 143, 1731-1742 (1993).
16. Ma, X.-J. et al. Gene expression profiles of human breast cancer progression. Proc. Natl. Acad. Sci. 100, 5974-5979 (2003).
17. Porter, D. et al. Molecular markers in ductal carcinoma in situ of the breast. Mol. Cancer Res. 1, 362-75 (2003).
18. Petridis, C. et al. Genetic predisposition to ductal carcinoma in situ of the breast. Breast Cancer Res. 18, 22 (2016).
19. Martelotto, L. G. et al. Whole-genome single-cell copy number profiling from formalin-fixed paraffin-embedded samples. Nat Med 23, 376-385 (2017).
20. Casasent, A. K. et al. Multiclonal Invasion in Breast Tumors Identified by Topographic Single Cell Sequencing. Cell 172, 205-217 e12 (2018).
21. Cowell, C. F. et al. Progression from ductal carcinoma in situ to invasive breast cancer: revisited. Mol Oncol 7, 859-869 (2013).
22. Nelson, C. M. & Bissell, M. J. Of extracellular matrix, scaffolds, and signaling: tissue architecture regulates development, homeostasis, and cancer. Annu. Rev. Cell Dev. Biol. 22, 287-309 (2006).
23. Hu, M. et al. Regulation of in situ to invasive breast carcinoma transition. Cancer Cell 13, 394-406 (2008).
24. Sirka, O. K., Shamir, E. R. & Ewald, A. J. Myoepithelial cells are a dynamic barrier to epithelial dissemination. J. Cell Biol. 217, 3368-3381 (2018).
25. Barsky, S. H. & Karlin, N. J. Myoepithelial Cells: Autocrine and Paracrine Suppressors of Breast Cancer Progression. J. Mammary Gland Biol. Neoplasia 10, 249-260 (2005).
26. Man, Y.-G. & Sang, Q.-X. A. The significance of focal myoepithelial cell layer disruptions in human breast tumor invasion: a paradigm shift from the “protease-centered” hypothesis. Exp. Cell Res. 301, 103-118 (2004).
27. Gudjonsson, T., Adriance, M. C., Sternlicht, M. D., Petersen, O. W. & Bissell, M. J. Myoepithelial Cells: Their Origin and Function in Breast Morphogenesis and Neoplasia. J. Mammary Gland Biol. Neoplasia 10, 261-272 (2005).
28. Lagace, R., Grimaud, J. A., Schurch, W. & Seemayer, T. A. Myofibroblastic stromal reaction in carcinoma of the breast: variations of collagenous matrix and structural glycoproteins. Virchows Arch A Pathol Anat Histopathol 408, 49-59 (1985).
29. Polyak, K. & Kalluri, R. The Role of the Microenvironment in Mammary Gland Development and Cancer. Cold Spring Harb. Perspect. Biol. 2, a003244-a003244 (2010).
30. Provenzano, P. P., Inman, D. R., Eliceiri, K. W., Trier, S. M. & Keely, P. J. Contact guidance mediated three-dimensional cell migration is regulated by Rho/ROCK-dependent matrix reorganization. Biophys J 95, 5374-5384 (2008).
31. Egeblad, M., Rasch, M. G. & Weaver, V. M. Dynamic interplay between the collagen scaffold and tumor evolution. Curr Opin Cell Biol 22, 697-706 (2010).
32. Levental, K. R. et al. Matrix crosslinking forces tumor progression by enhancing integrin signaling. Cell 139, 891-906 (2009).
33. Nguyen-Ngoc, K.-V. et al. ECM microenvironment regulates collective migration and local dissemination in normal and malignant mammary epithelium. Proc. Natl. Acad. Sci. 109, E2595-E2604 (2012).
34. Carey, S. P., Martin, K. E. & Reinhart-King, C. A. Three-dimensional collagen matrix induces a mechanosensitive invasive epithelial phenotype. Sci. Rep. 7, 42088 (2017).
35. Linnemann, J. R. et al. Quantification of regenerative potential in primary human mammary epithelial cells. Development 142, 3239-3251 (2015).
36. Gudjonsson, T. et al. Normal and tumor-derived myoepithelial cells differ in their ability to interact with luminal breast epithelial cells for polarity and basement membrane deposition. J Cell Sci 115:39-50, (2002).
37. Debnath, J. & Brugge, J. S. Modelling glandular epithelial cancers in three-dimensional cultures. Nat. Rev. Cancer 5, 675-688 (2005).
38. Portillo-Lara, R. & Annabi, N. Microengineered cancer-on-a-chip platforms to study the metastatic microenvironment. Lab Chip 16, 4063-4081 (2016).
39. Santagata, S. et al. Taxonomy of breast cancer based on normal cell phenotype predicts outcome. J Clin Invest 124, 859-870 (2014).
40. Burdall, S. E., Hanby, A. M., Lansdown, M. R. & Speirs, V. Breast cancer cell lines: friend or foe? Breast Cancer Res. 5, 89 (2003).
41. Qu, Y. et al. Evaluation of MCF10A as a Reliable Model for Normal Human Mammary Epithelial Cells. PLOS One 10, e0131285 (2015).
42. Sachs, N. et al. A Living Biobank of Breast Cancer Organoids Captures Disease Heterogeneity. Cell 172, 373-386 e10 (2018).
43. Eirew, P. et al. A method for quantifying normal human mammary epithelial stem cells with in vivo regenerative ability. Nat Med 14, 1384-1389 (2008).
44. Wozniak, M. A. & Keely, P. J. Use of three-dimensional collagen gels to study mechanotransduction in T47D breast epithelial cells. Biol. Proced. Online 7, 144-61 (2005).
45. Hu, Y. & Smyth, G. K. ELDA: extreme limiting dilution analysis for comparing depleted and enriched populations in stem cell and other assays. J Immunol Methods 347, 70-78 (2009).
46. Rattan, B. et al. The Immunohistochemical Expression of the Oestrogen Receptor (ER), HER-2/NEU and Cytokeratin 8/18 and 5/6 in Invasive Breast Carcinoma. J Clin Diagn Res 6, 1495-1498 (2012).
47. Bell, J., Walsh, S., Nusrat, A. & Cohen, C. Zonula occludens-1 and Her-2/neu expression in invasive breast carcinoma. Appl Immunohistochem Mol Morpho/11, 125-129 (2003).
48. Matsukita, S. et al. Expression of mucins (MUC1, MUC2, MUC5AC and MUC6) in mucinous carcinoma of the breast: comparison with invasive ductal carcinoma. Histopathology 42, 26-36 (2003).
49. Friedl, P. & Gilmour, D. Collective cell migration in morphogenesis, regeneration and cancer. Nat Rev Mol Cell Biol 10, 445-457 (2009).
50. Gudjonsson, T. et al. Isolation, immortalization, and characterization of a human breast epithelial cell line with stem cell properties. Genes Dev 16, 693-706 (2002).
51. Zhou, H. & Kramer, R. H. Integrin engagement differentially modulates epithelial cell motility by RhoA/ROCK and PAK1. J Biol Chem 280, 10624-10635 (2005).
52. Yu, W. et al. Involvement of RhoA, ROCK I and myosin II in inverted orientation of epithelial polarity. EMBO Rep 9, 923-929 (2008).
53. Winkler, J., Abisoye-Ogunniyan, A., Metcalf, K. J. & Werb, Z. Concepts of extracellular matrix remodelling in tumour progression and metastasis. Nat. Commun. 11, 5120 (2020).
54. Provenzano, P. P. et al. Collagen reorganization at the tumor-stromal interface facilitates local invasion. BMC Med 4, 38 (2006).
55. Brownfield, D. G. et al. Patterned collagen fibers orient branching mammary epithelium through distinct signaling modules. Curr Biol 23, 703-709 (2013).
56. Friedl, P. & Wolf, K. Tube travel: the role of proteases in individual and collective cancer cell invasion. Cancer Res 68, 7247-7249 (2008).
57. Kessenbrock, K., Plaks, V. & Werb, Z. Matrix metalloproteinases: regulators of the tumor microenvironment. Cell 141, 52-67 (2010).
58. Friedl, P., Locker, J., Sahai, E. & Segall, J. E. Classifying collective cancer cell invasion. Nat Cell Biol 14, 777-783 (2012).
59. McCart Reed, A. E., Kutasovic, J. R., Lakhani, S. R. & Simpson, P. T. Invasive lobular carcinoma of the breast: morphology, biomarkers and 'omics. Breast Cancer Res 17, 12 (2015).
60. Schipper, K. et al. Rebalancing of actomyosin contractility enables mammary tumor formation upon loss of E-cadherin. Nat. Commun. 10, 3800 (2019).
61. Kalluri, R. The biology and function of fibroblasts in cancer. Nat. Rev. Cancer 16, 582-598 (2016).
62. Hanahan, D. & Weinberg, R. A. Hallmarks of cancer: the next generation. Cell 144, 646-674 (2011).
63. Linnemann, J. R., Meixner, L. K., Miura, H. & Scheel, C. H. An Organotypic 3D Assay for Primary Human Mammary Epithelial Cells that Recapitulates Branching Morphogenesis. in 125-137 (2017). doi: 10.1007/978-1-4939-7021-6_9.
64. Geraldo, S., Simon, A. & Vignjevic, D. M. Revealing the Cytoskeletal Organization of Invasive Cancer Cells in 3D. J. Vis. Exp. (2013) doi: 10.3791/50763.
65. Buchmann, B. et al. Mechanical plasticity of the ECM directs invasive branching morphogenesis in human mammary gland organoids. bioRxiv (2019) doi: 10.1101/860015.
66. Breunig, C. T. et al. One step generation of customizable gRNA vectors for multiplex CRISPR approaches through string assembly gRNA cloning (STAgR). PLOS One 13, e0196015 (2018).
67. Cheung, K. J. et al. Collective invasion in breast cancer requires a conserved basal epithelial program. Cell 155, 1639-51 (2013) doi: 10.1016/j.cell.2013.11.029.

1. An in vitro method of generating cells capable of differentiating to a multicellular organoid unit that morphologically and/or functionally recapitulates invasive and/or ductal cell growth, comprising the steps of:
(i) Providing luminal progenitor (LP) cells,
(ii) culturing said luminal progenitor (LP) cells in a collagen gel in a culture medium for at least 5 days,
wherein said culture medium comprises
one or more growth factor(s),
one or more inhibitor(s) of cell contractility and
one or more serum component or serum substitute,
(iii) determining whether a multicellular organoid unit is formed in step (ii).

2. The in vitro method according to claim 1, wherein determining whether a multicellular organoid unit is formed is by determining whether an invasive organoid or a ductal structure or one or more branch-point(s) is/are comprised in said multicellular organoid unit.

3. The in vitro method according to claim 1 or claim 2, wherein the provided luminal progenitor (LP) cells are human primary luminal cells or human mammary luminal progenitor cells, preferably healthy human primary luminal cells or healthy human mammary luminal progenitor cells.

4. The in vitro method according to any one of the preceding claims, wherein the one or more growth factor(s) of the culture medium is a ligand of the ErbB receptor family, preferably the epidermal growth factor (EGF) or an analogue thereof.

5. The in vitro method according to any one of the preceding claims, wherein the one or more inhibitor(s) of cell contractility of the culture medium is a Rho-kinase (ROCK) inhibitor, preferably Y-27632 or thiazovivin, or a myosin inhibitor, preferably para-amino blebbistatin.

6. The in vitro method according to any one of the preceding claims, wherein the culture medium further comprises one or more selected from the group consisting of N-acetylcysteine, neuregulin 1, a vitamin, preferably nicotinamide, an antibiotic, a fibroblast growth factor (FGF), preferably FGF7 or FGF10, a MAP kinase inhibitor, preferably SB202190, a supplement and a buffer.

7. The in vitro method according to any one of the preceding claims, wherein the provided luminal progenitor (LP) cells are genetically modified, preferably wherein one or more gene(s) are knocked-out in the provided luminal progenitor (LP) cells.

8. The in vitro method according to any one of the preceding claims, wherein the collagen concentration of the collagen gel is in a range of about 0.5 mg/ml to about 3 mg/ml, preferably of about 0.8 mg/ml to about 2.0 mg/ml, more preferably of about 1.0 mg/ml to about 1.5 mg/ml.

9. The in vitro method according to any one of the preceding claims, wherein the multicellular organoid unit morphologically and/or functionally recapitulates low-grade invasive processes of mammary cancer, preferably of low-grade carcinoma of no special type (NST).

10. A method of screening for an anti-migratory drug, comprising the following steps:
a) Bringing a multicellular organoid unit obtained by the method of any one of the preceding claims into contact with a compound suspected of being an anti-migratory drug,
b) determining whether or not said compound elicits a cellular response in the multicellular organoid unit,
with the proviso that when said compound elicits a cellular response compared to a reference state, said compound is an anti-migratory drug.

11. The method of screening according to claim 10, wherein the cellular response is selected from the group consisting of cell elongation arrest, cell proliferation arrest, growth arrest, apoptosis, necrosis, DNA damage, inhibition of differentiation, migration arrest, and changes in the morphology of cells, preferably cell elongation arrest.

12. A culture medium comprising:
one or more growth factor(s),
one or more inhibitor(s) of cell contractility,
one or more serum component or serum substitute, and
further one or more of the group consisting of N-acetylcysteine, neuregulin 1, a vitamin, preferably nicotinamide, an antibiotic, a fibroblast growth factor (FGF), preferably FGF7 or FGF10, a MAP kinase inhibitor, preferably SB202190, a supplement and a buffer.

13. The culture medium according to claim 12, wherein the one or more growth factor(s) is a ligand of the ErbB receptor family, preferably the epidermal growth factor (EGF) or an analogue thereof.

14. The culture medium according to claim 12 or 13, wherein the one or more inhibitor(s) of cell contractility is a Rho-kinase (ROCK) inhibitor, preferably Y-27632 or thiazovivin, or a myosin inhibitor, preferably para-amino blebbistatin.

15. Use of a culture medium according to any one of claims 12 to 14 in a method according to any one of claims 1 to 11.


SEQUENCE LISTING (4 sequences)

SEQ ID NO: 1 - DNA, 20 bp, synthetic construct
  source 1..20: mol_type = other DNA
  misc_feature 1..20: note = sgRNA sequence for targeting CDH1
ataataaagacaccaacagg

SEQ ID NO: 2 - DNA, 20 bp, synthetic construct
  source 1..20: mol_type = other DNA
  misc_feature 1..20: note = sgRNA sequence for targeting CDH1
ttagaagcttgttgacaccg

SEQ ID NO: 3 - DNA, 20 bp, synthetic construct
  source 1..20: mol_type = other DNA
  misc_feature 1..20: note = primer sequence
gctgtctggctaggttggac

SEQ ID NO: 4 - DNA, 20 bp, synthetic construct
  misc_feature 1..20: note = primer sequence
  source 1..20: mol_type = other DNA
gatccagcatgggttgacca